(19) United States
(12) Patent Application Publication
(10) Pub. No.: US 2021/0321883 A1
(43) Pub. Date: Oct. 21, 2021

(54) MODULAR AMBULATORY HEALTH STATUS AND PERFORMANCE TRACKING SYSTEM

(71) Applicant: MEDTRONIC, INC., Minneapolis, MN (US)

(72) Inventors: Scott T. Mcmahan, Cincinnati, OH (US); Mark J. Phelps, Tempe, AZ (US); Randal Schulhauser, Phoenix, AZ (US)

(73) Assignee: MEDTRONIC, INC., Minneapolis, MN (US)

(21) Appl. No.: 16/849,524

(22) Filed: Apr. 15, 2020

(51) Int. Cl. A61B 5/0205; A61B 5/145; A61B 5/00; A61B 5/024; A61B 5/11

(52) U.S. Cl. CPC A61B 5/02055; A61B 5/14503; A61B 5/6867; A61B 5/02416; A61B 2560/0443; A61B 5/1114; A61B 2562/0219; A61B 2562/0271; A61B 5/746

(57) ABSTRACT

A system comprising at least one implanted sensor device comprising one or more continuous sensors to detect a first modular set of biometrics of a subject. A system includes a computing device that receives the first modular set of biometrics over a period of time and receives a second modular set of biometrics for the subject. The computing device selectively serves a graphical user interface configured to present one or more of the first modular set and second modular set of biometrics and normalizes at least a portion of the first modular set and second modular set of biometrics over a period of time. The device generates a score indicative of a state of a biological system of the subject corresponding to a portion of biometrics of the first modular set and second modular set of biometrics and causes the score to be displayed via an electronic device.

### FIELD

The present technology is generally related to a modular ambulatory health status and performance tracking system.

### BACKGROUND

Movement is essential to life. A small subset of the population, however, undeniably does it much better than the rest of us. Intricately understanding how athletes or other people perform at a significantly higher level than everyone else would provide insight into how to optimize and scale physical and mental well-being and toughness to the masses.

In hospitals today, a patient is valuated on a set of biometrics sometimes referred to as “standard of care” biometrics. A patient may not be able to go home, in some instances, if they have a temperature. In other instances, a patient's glucose may be elevated, and potentially requiring treatment. Biometrics associated with infections may be evaluated when determining the health of a patient and/or determining whether to discharge the patient. The “standard of care” biometrics are medical-grade biometrics.

Currently, consumers have access to activity monitors. However, these activity monitors do not produce medical grade biometrics.

### SUMMARY

The techniques of this disclosure generally relate to a modular eco-system that is adapted to provide medical-grade biometrics for one or more of diagnosing a disease by a medical facility or practitioner, tracking by a consumer their health status in real-time and human performance tracking.

In one aspect, the present disclosure provides a system comprising at least one implanted sensor device comprising one or more continuous sensors to detect a first modular set of biometrics of a subject. The system includes an assessment system with a computing device and a computer-readable storage medium comprising one or more programming instructions that, when executed, cause the computing device to receive the first modular set of biometrics over a period of time and receive a second modular set of biometrics for the subject. The computing device selectively serves a graphical user interface configured to present one or more of the first modular set and second modular set of biometrics and normalizes at least a portion of the first modular set and second modular set of biometrics over a period of time. The device generates a score indicative of a state of a biological system of the subject corresponding to a portion of biometrics of the first modular set and second modular set of biometrics and causes the score to be displayed via an electronic device.

In another aspect, the disclosure provides a method comprising sensing, by at least one implanted sensor device comprising one or more continuous sensors, a first modular set of biometrics of a subject. The method includes, by at least one processor: receiving the first modular set of biometrics from the one or more continuous sensors over a period of time; receiving a second modular set of biometrics associated with the subject; selectively serving a graphical user interface configured to present one or more of the first modular set of biometrics and the second modular set of biometrics; normalizing at least a portion of the first modular set of biometrics and the second modular set of biometrics over a period of time; generating a score indicative of a state of a biological system of the subject corresponding to the at least a portion of biometrics of the first modular set of biometrics and the second modular set of biometrics; and causing the score to be displayed via a client electronic device.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1A is a block diagram that illustrates an embodiment of an eco-system with a medical health status system, consumer ambulatory health status system and human performance tracking system.

FIG. 1B is a block diagram that illustrates an embodiment of a combined modular ambulatory health status and performance tracking system.

FIG. 2 is a perspective view of a simplified representation of a biometric sensor device as deployed for use on the skin of a patient with an implanted

FIG. 3A is a block diagram of an embodiment of various components sensed by an implanted biometric sensor device.

FIG. 3B is a block diagram of an embodiment of various components generated by an implanted continuous infection detection sensor device.

FIG. 3C is a block diagram of an embodiment of various components sensed by an implanted continuous basic metabolic panel sensor device.

FIG. 3D is a block diagram of an embodiment of a component generated by an implanted continuous glucose sensor device.

FIG. 4 is a block diagram of an embodiment of various components sensed by a digital column scale.

FIG. 5 is a block diagram of an embodiment of various components sensed by a point of care (PoC) lab-on-chip device associated with standard of care biometrics.

FIG. 6 is a block diagram of an embodiment of various components generated by a medical laboratory associated with standard of care biometrics.

FIG. 7 is a block diagram of an embodiment of various components generated by a medical laboratory associated with standard of care biometrics.

FIG. 8 is a block diagram of an embodiment of a user electronic device interfacing with the modular ambulatory health status and performance tracking system to collect standard of care biometrics.

FIG. 9 is a block diagram of the health tracking module.

FIG. 10 is a block diagram of the human performance tracking module.

FIG. 11 is a diagram of an embodiment of various components of a graphical user interface (GUI) for standard of care biometric navigation.

FIG. 12A is a diagram of an embodiment of various components of a GUI for identifying and/or selecting various components sensed by an implanted biometric device.

FIG. 12B is a diagram of an embodiment of various components of a GUI for displaying in real-time biometrics of the various components sensed by an implanted biometric sensor device.

FIG. 12C is a diagram of an embodiment of various components of a GUI for displaying a trending graph of various components of the implanted biometric sensor device.

FIG. 13A is a diagram of an embodiment of a GUI for identifying and/or selecting various components sensed by a point of care (PoC) lab-on-chip device associated with standard of care biometrics.

FIG. 13B is a diagram of an embodiment of a GUI for selectively identifying value ranges for various components sensed by a point of care (PoC) lab-on-chip device associated with standard of care biometrics.

FIG. 14A is a diagram of an embodiment of a GUI for displaying a graph of a computed heath score using selected biometrics.

FIG. 14B is a diagram of an embodiment of a GUI for displaying a graph of an example computed heath score using selected biometrics.

FIG. 14C is a diagram of an embodiment of a GUI for displaying a graph of another example of a computed health score using selected biometrics.

FIG. 15 is a diagram of an embodiment of a GUI for displaying infection biometric data.

FIG. 16 is a flowchart of an embodiment of a method for human performance tracking.

FIG. 17 is a flowchart of an embodiment of a method for training with nonfunctional overreaching (NFOR), functional overreaching (FOR), and overtraining syndrome (OTS) analysis.

FIG. 18 is a flowchart of an embodiment of a method for sleep tracking.

FIG. 19 is a flowchart of an embodiment of a method for training and recovery tracking.

FIG. 20A is a diagram of an embodiment of a mobile device displaying a human performance tracking graphical user interface.

FIG. 20B is a diagram of an embodiment of a mobile device displaying another human performance tracking graphical user interface.

FIG. 21 depicts an example of internal hardware that may be included in any of the electronic components of an electronic device.

### DETAILED DESCRIPTION

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting.

In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other. Generally, similar spatial references of different aspects or components indicate similar spatial orientation and/or positioning, i.e., that each “first end” is situated on or directed towards the same end of the device. Further, the use of various spatial terminology herein should not be interpreted to limit the various location techniques or orientations for identifying objects or machines.

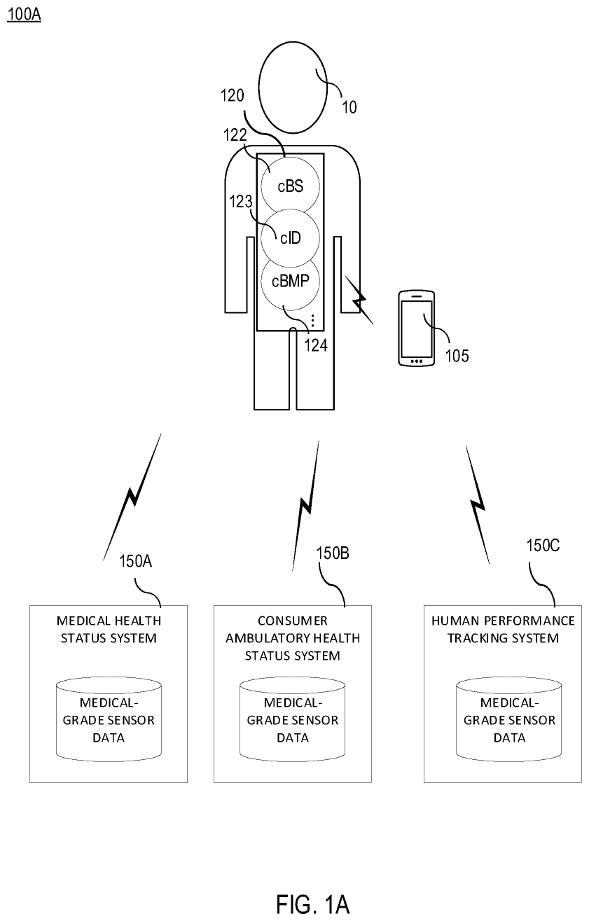

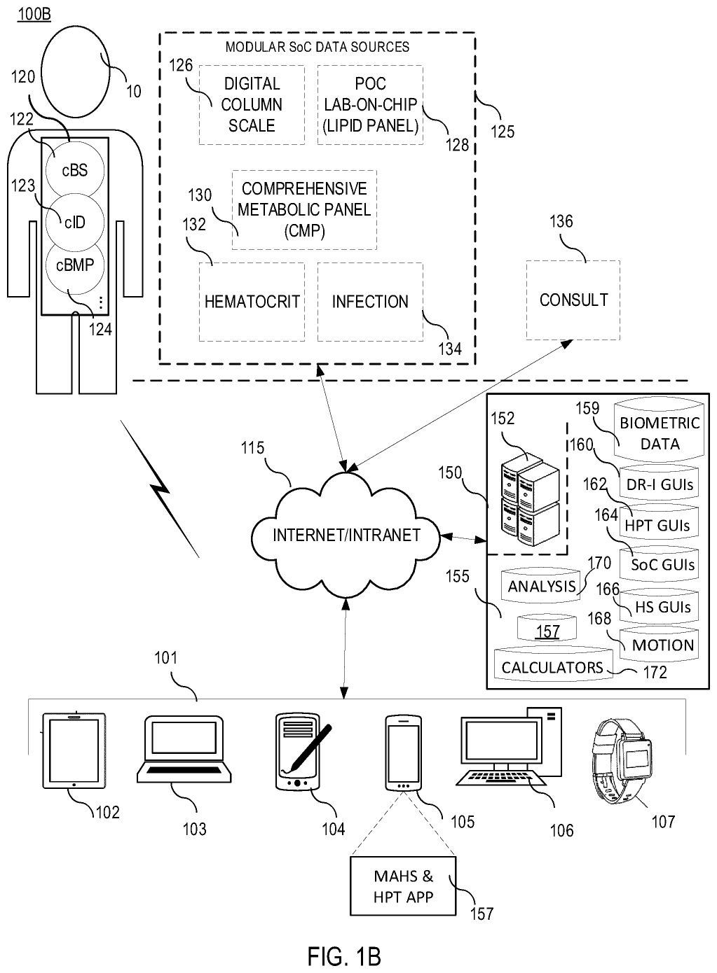

An “electronic device” or a “computing device” refers to a device or system that includes a processor and memory. Each device may have its own processor and/or memory, or the processor and/or memory may be shared with other devices as in a virtual machine or container arrangement. The memory can contain or receive programming instructions that, when executed by the processor, cause the electronic device to perform one or more operations according to the programming instructions. Examples of electronic devices include personal computers, servers, mainframes, virtual machines, containers, cameras, tablet computers, laptop computers, media players and the like. Electronic devices also may include appliances and other devices that can communicate in an Internet-of-things arrangement. In a client-server arrangement, the client device and the server are electronic devices, in which the server contains instructions and/or data that the client device accesses via one or more communications links in one or more communications networks. In a virtual machine arrangement, a server may be an electronic device, and each virtual machine or container also may be considered an electronic device. In the discussion above, a client device, server device, virtual machine or container may be referred to simply as a “device” for brevity. Additional elements that may be included in electronic devices are discussed in the context of FIGS. 1A-1B.

The terms “processor” and “processing device” refer to a hardware component of an electronic device that is configured to execute programming instructions. Except where specifically stated otherwise, the singular terms “processor” and “processing device” are intended to include both single-processing device embodiments and embodiments in which multiple processing devices together or collectively perform a process.

The terms “memory,” “memory device,” “data store,” “data storage facility” and the like each refer to a tangible and non-transitory device on which computer-readable data, programming instructions or both are stored. Except where specifically stated otherwise, the terms “memory,” “memory device,” “data store,” “data storage facility” and the like are intended to include single device embodiments, embodiments in which multiple memory devices together or collectively store a set of data or instructions, as well as individual sectors within such devices.

In this document, the terms “communication link” and “communication path” mean a wired or wireless path via which a first device sends communication signals to and/or receives communication signals from one or more other devices. Devices are “communicatively connected” if the devices are able to send and/or receive data via a communication link. “Electronic communication” refers to the transmission of data via one or more signals between two or more electronic devices, whether through a wired or wireless network, and whether directly or indirectly via one or more intermediary devices.

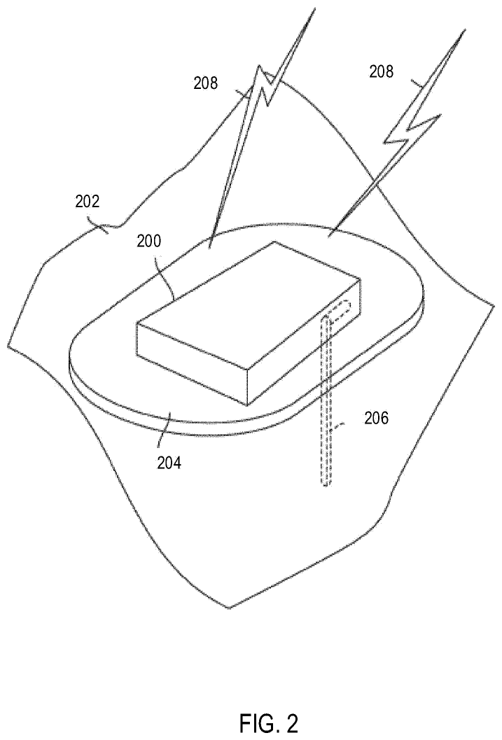

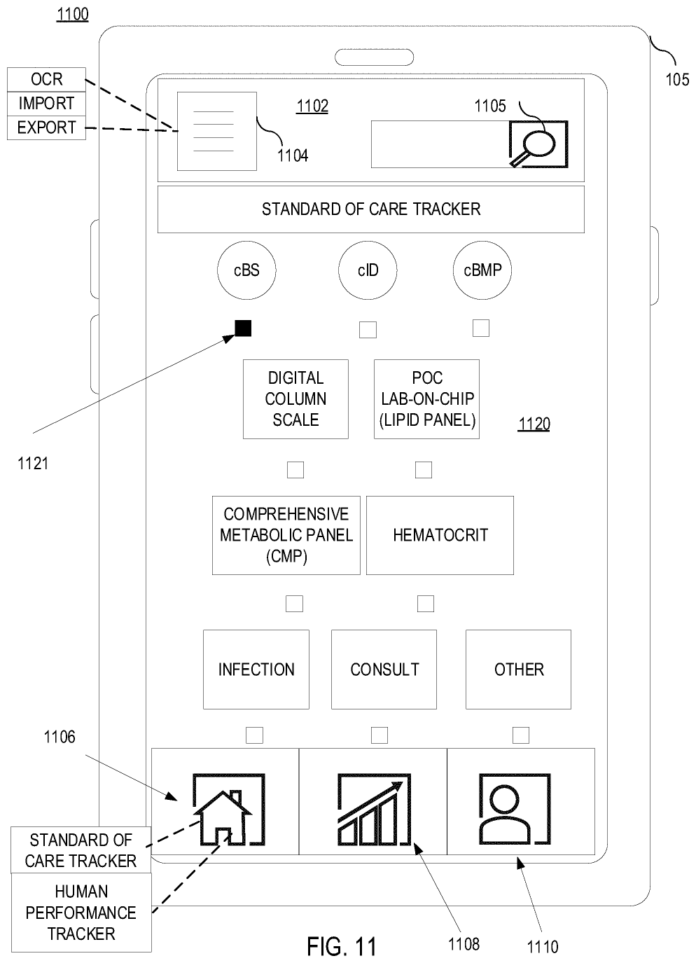

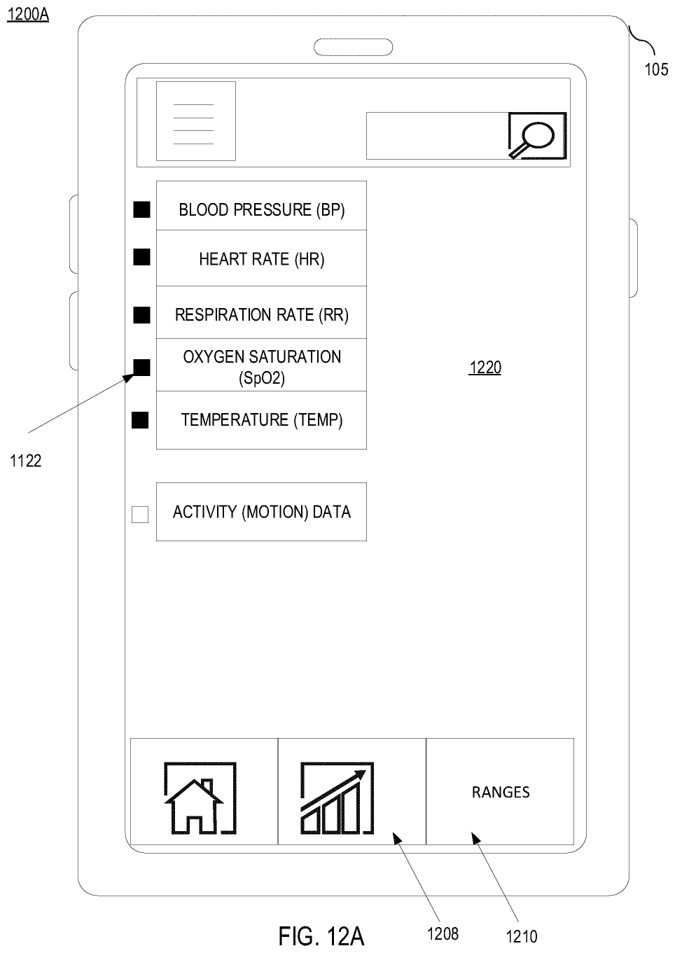

FIG. 1A is a block diagram that illustrates an embodiment of an eco-system 100A with a medical health status system 150A, consumer ambulatory health status system 150B and human performance tracking system 150C. The medical health status system 150A, consumer ambulatory health status system 150B and human performance tracking system 150C may each communicate with a modular implanted sensor system 120 with one or more transcutaneously sensor elements 206 (FIG. 2) configured to be transcutaneously insertable into a biological system. The modular implanted sensor system 120 may include one or more sensor devices such as one or more of sensor devices 122, 123 and 124. The modular implanted sensor system 120 may be configured to communicate with a user electronic device such as a smart phone 105. The modular implanted sensor system 120 may be configured to selectively communicate medical-grade biometrics to one or more of the medical health status system 150A, consumer ambulatory health status system 150B and/or the human performance tracking system 150C using the smart phone or other user electronic device as will be described in more detail in FIG. 1B. The medical health status system 150A may interface with the graphical user interface 1100 of FIG. 11 describe later. The biometric data is shown stored in a database in each of the medical health status system 150A, consumer ambulatory health status system 150B and the human performance tracking system 150C, for example.

The medical health status system 150A may use medical-grade biometric data from the modular implanted sensor system 120 for the purposes of a medical diagnosis by a doctor, medical practitioner or healthcare facility. The consumer ambulatory health status system 150B may use medical-grade biometric data from the modular implanted sensor system 120 for the purposes of providing to the subject, health status information in real-time that may be used by the subject to make improvements in their health. Furthermore, the health status information may be an early indicator or alert of an imminent health crisis, by way of non-limiting example. The health status information may be an early indicator a health status improvement, by way of non-limiting example

The human performance tracking system 150C may use medical-grade biometric data from the modular implanted sensor system 120 for the purposes of providing to a subject, human performance information in real-time that may be used by the subject to make improvements in their performance, such as for athletic training, rehabilitation, and physical therapy.

A modular system with a combined modular ambulatory health status and performance tracking system 100B will be described in relation to FIG. 1B. However, the modular feature of system 100B may allow system 100B to function for only health status tracking, only human performance or a combination of both health status and human performance tracking. The health status tracking may be based on normal ranges of biometrics associated with a population. The normal ranges of biometrics may vary based on age, sex, race and geographic location. The human performance tracking may be based on the subject's performance abilities from a disabled perspective up to the performance ability of an elite athlete. The human performance ability may be based on an “ideal” model. For example, if the subject plays basketball, the “ideal” model would be a function of performance metrics for an “ideal” basketball player, such as for jumping, running, etc. On the other hand, if the subject is a runner, the “ideal” model would be a function of performance metrics for an “ideal” runner or sprinter.

FIG. 1B is a block diagram that illustrates an embodiment of a combined modular ambulatory health status and performance tracking system 100B. The system 100B may include a user electronic device 101 which may, for example, include a tablet 102, a laptop 103, a personal digital assistant (PDA) 104, a smart phone 105, a personal computer 106 and a smart watch 107. The user electronic device 101 may include other electronic devices with display devices which may be head mounted or body worn. A head mounted display (HMD) device may be integrated into glasses or goggles.

The user electronic device 101 may include a global positioning system and/or inertial navigation system for estimating a geographical location of the user electronic device 101, such estimation of geographical location being well known in the art. The inertial navigation system may include accelerometers, magnetometer and/or gyroscopes. The inertial navigation system may include an inertial measurement unit (IMU).

The system 100B may include a modular implanted sensor system 120. The modular implanted sensor system 120 may include one or more sensor devices, only sensor devices 122, 123 and 124 are shown in FIG. 1B. By way of non-limiting example, the implanted sensor system 120 may include a biometric sensor device 122 such as a LINQ™ II implantable cardiac sensor by Medtronic™, Inc. The biometric sensor device 122 may communicate over a communication path using BLUETOOTH LOW ENERGY (BLE) protocols, WIFI, near field communications (NFC), tissue conductance communication (TCC), telemetry or other wireless communication protocols. The biometric sensor device 122 may include a system on-a-chip BLE modules. The biometric sensor device 122 may have direct connectivity to a user electronic device 101. TCC is an intrabody communication protocol which allows implantable devices to communicate with each other. Any communication unit may include a transmitter and receiver.

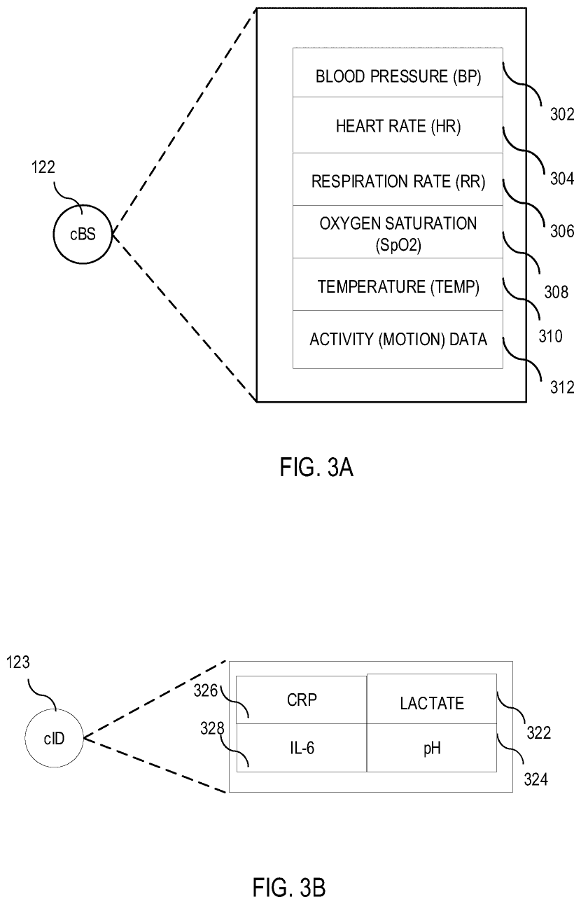

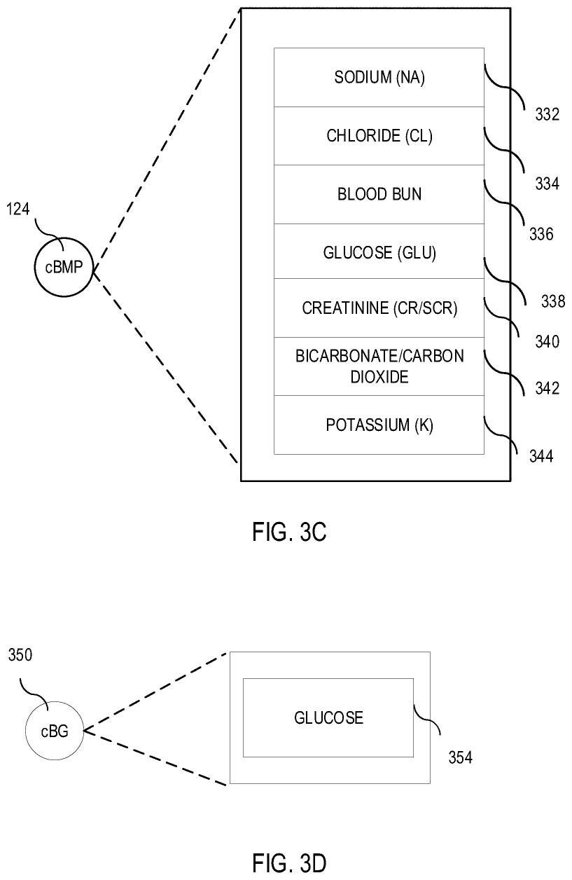

The modular implanted sensor system 120 may include a continuous basic metabolic panel sensor device 124, for example. The modular implanted sensor system 120 may include a continuous infection detection sensor device 123. The modular implanted sensor system 120 may include other sensor devices, such a glucose sensor device 350 (FIG. 3D). Nonetheless, the one or more sensor devices may include a sensor element 206 (FIG. 2) configured to be transcutaneously insertable into a biological system such as into the skin 202 (FIG. 2) of a subject 10. The term “implanted” as used herein is used to describe a sensor which has one or more portions inserted into the biological system of the user such as through the skin. In some embodiment, a sensor element 206 pierces the skin which other parts of the sensor remaining external to the biological system.

The term “modular” may be defined as “selectively integrated” and/or “selective functionality.” In some embodiments, a subject using the system 100B may not have need for a continuous infection detection sensor device 123. Furthermore, the continuous infection detection sensor device 123 may be needed only temporarily such as after a surgery or other condition while outside of a hospital or medical facility.

The user electronic device 101 may access a computing system 150 via a communication network 115 such as an intranet or the Internet. The network communications may use wired or wireless communication media and related protocols associated with the platform type of the electronic device 101. The electronic device 101 may communicate with the computing system 150 using known electronic communications. The computing system 150 may be a combination of systems 150A and 150B, in some embodiments. The modular implanted sensor system 120 may also communicate with the user electronic device 101. In some embodiments, the modular implanted sensor system 120 may generate a first modular set of sensor biometric data that is communicated to the user electronic device 101. In turn, the user electronic device 101 may communicate, via the communication network 115, the first modular set of sensor biometric data to the computing system 150 for further review and analysis. The computing system 150 is the assessment system having programming instructions configured to determining a score associated with a biological system of a subject using medical-grade biometrics. One or more subsets of the first modular set of sensor biometric data may be derived by the modular standard of care data sources 125. By way of non-limiting example, if the implant sensor system 120 does not include a continuous basic metabolic panel, then such subset of biometric data may be obtained using the modular standard of care data sources 125.

The user electronic device 101 may have a web browser and/or a modular ambulatory health status (MAHS) and human performance tracking (HPT) application 157 downloaded onto the electronic device 101. The MAHS and HPT application 157 may include programming functions configured to provide the user access to the computing system 150, for example. In the illustration of FIG. 1B, the MAHS and HPT application 157 is represented as being downloaded on smart phone 105 from the computing system 150. To avoid crowding, the MAHS and HPT application 157 includes a plurality of graphical user interfaces (GUIs), such as one or more GUIs to display the modular standard of care biometric data, as will be described in more detail below. The MAHS and HPT application 157 may be downloaded and installed on other electronic devices 101, but to minimize crowding in the figure, only one application shown.

The MAHS and HPT application 157 provided by the computing system 150 may include programming instructions configured to allow a first modular set of biometric data to be received and stored from the modular implant sensor system 120 into a biometric data database 159. The MAHS and HPT application 157, via the computing system 150, may include programming instructions configured to allow a second modular set of medical-grade biometric data to be received and stored by one or more of the user electronic device 105 and/or the computing system 150. The second modular set of biometric data may be stored in the biometric data database 159. The second modular set of biometric data may include data from one or more sources of the modular standard of care data sources 125. The modular standard of care data sources 125 are represented in dashed lines to represent that these sources 125 are not part of the system 100B. Instead, the application 157 may include a communications interface and/or an application programming interface (API) to access a subject's medical biometrics data to receive or retrieve different subsets of medical-grade biometric data electronically over a communication path using the Internet/Intranet 115. In other embodiments, the subsets of medical-grade biometric data may be manually entered by the user and stored in the biometric data database 159, as new subset of biometric data becomes available.

By way of a non-limiting example, the second modular set of biometric data or one or more subsets of the biometric data of the second set may be generated as a result of a recent stay in a hospital or based on a recent annual physical. The second modular set of biometric data may include one or more subsets of biometric data associated with the first set of medical-grade biometric data. Overtime, any updates in biometric data, such as the second set of biometric data may be provided by medical laboratories. In an embodiment, the computing system 150 may interface with a hospital records, medical records or other medical-grade patient monitoring systems.

The second modular set of biometric data may include one or more subsets of biometric data such as from a digital column scale 126, a point of care (PoC) lab-on-chip device 128 for providing biometric data associated with a lipid panel, a comprehensive metabolic panel 130, hematocrit 132 and certain infection data 134. Either manually or automatically, one or more of the modular standard of care data sources 125 may provide a hardcopy or an electronic version of the second modular set of biometric data.

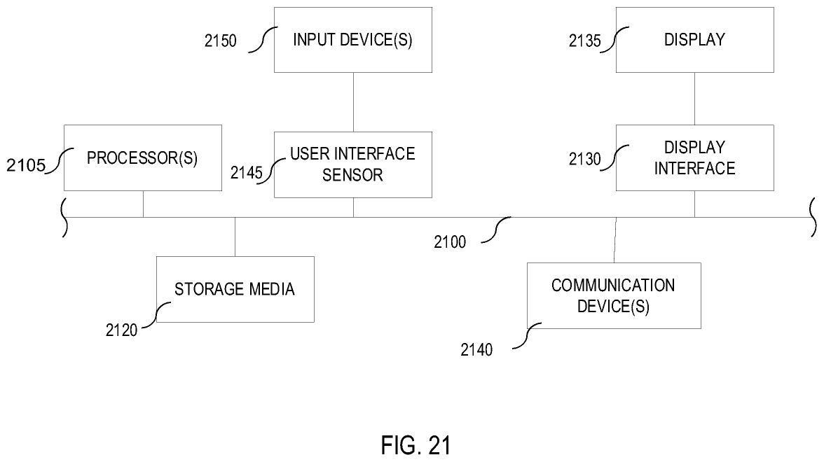

As illustrated in FIG. 1B, the computing system 150 may include a website, remote computing system or cloud computing system. The computing system 150 may include memory 155 having programming instructions stored thereon. The computing system 150 may include one or more servers or computing devices 152 configured to execute the programming instructions stored in memory 155. The server or computing device 152 will be described in more detail in relation to FIG. 21. The server or computing devices 152 may have web applications running thereon. The memory 155 may include a plurality of memory devices and/or databases.

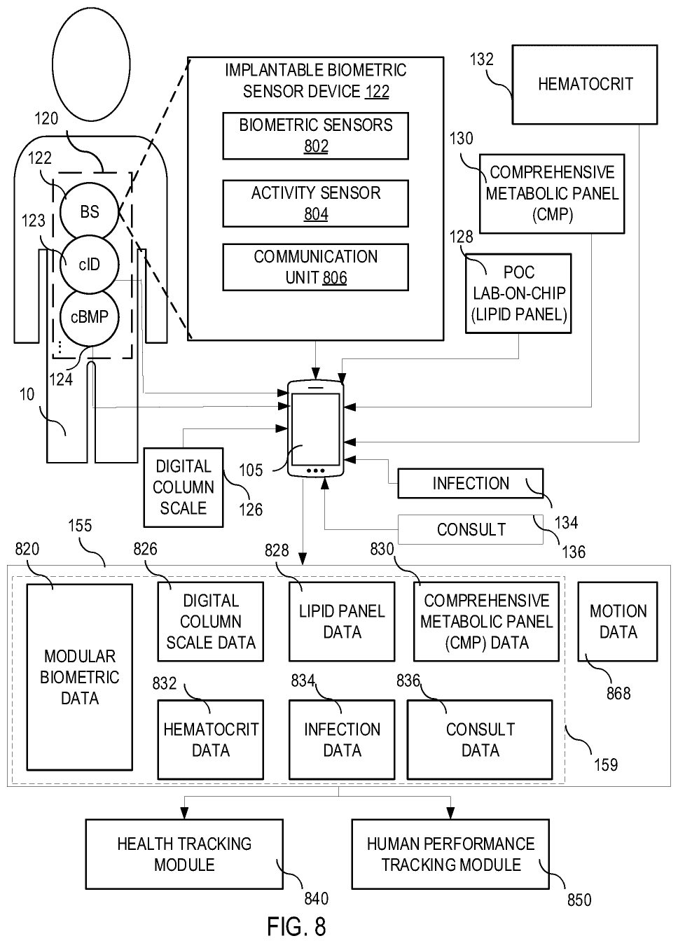

In some embodiments, the user electronic device 105 may communicate or receive data via a doctor or healthcare practitioner 136 that is stored as consult data 836 (FIG. 8). By way of non-limiting example, the doctor or healthcare practitioner 136 may select certain biometric data of the standard of care biometric data that may be given more weight or selected for specific analysis. The MAHS and HPT application 157 may include programming instructions for displaying doctor interface (DR-I) GUIs 160 to allow the doctor to select one or more of the standard of care biometric data for specific analysis or alerts, as will be described later. A doctor may set an alert level instruction for a score or biometric value for one or more biometrics. The system 150 may alert the doctor if any one or more of the scores or values of a biometric meets the alert level instruction.

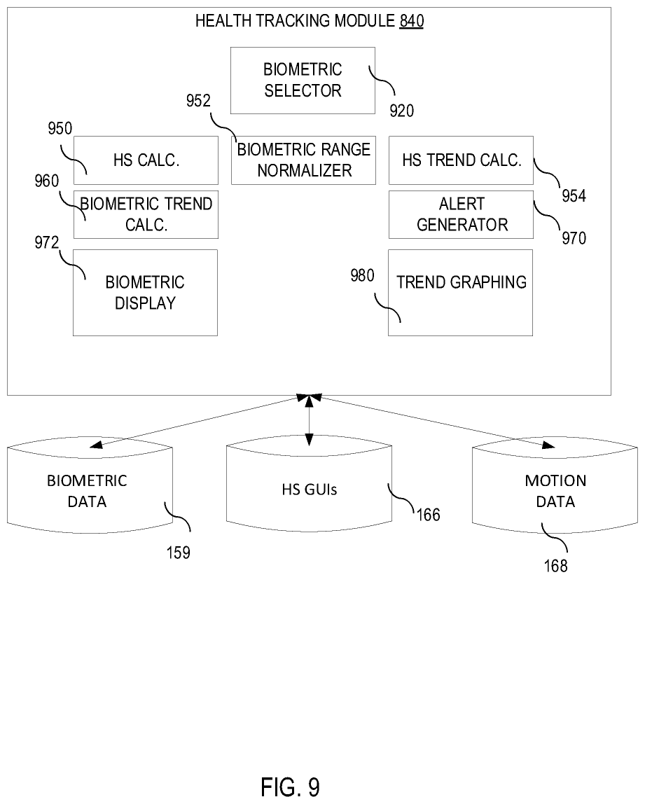

The memory 155 may include programming instructions for analysis 170 and calculations 172, as will be described in relation to FIGS. 9-10, 12B-12C, 14A-14C, 16-19 and 20B. The programming instructions for analysis 170 and calculations 172 when executed may cause a processor to execute machine learning algorithms that may use both supervised and unsupervised algorithms. The memory 155 may include programming instructions representative of machine learning algorithms to track, locate and predict human performance, predict wellness or predict reduction in a health status.

Common algorithms for performing classification processes by the machine learning algorithms may include a support vector machine (SVM), boosted and bagged decision trees, k-nearest neighbor, Naïve Bayes, Bayesian Belief probabilistic algorithms, and discriminant analysis. For example, Bayesian Belief probabilistic algorithms may be used with continuous metabolic panel sensor devices with specific application to glucose and potassium sensing machine learning algorithms.

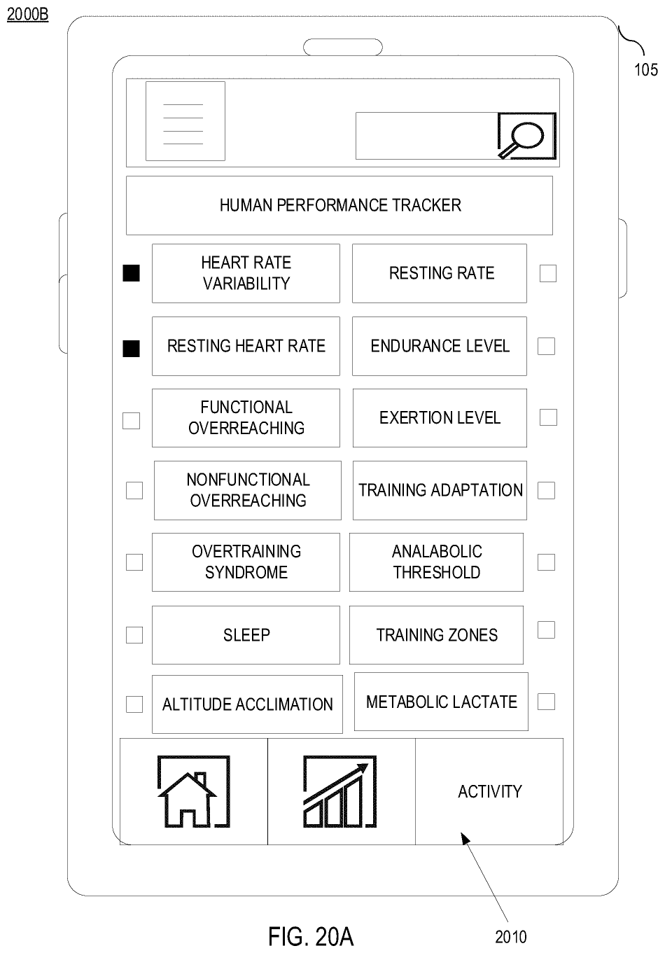

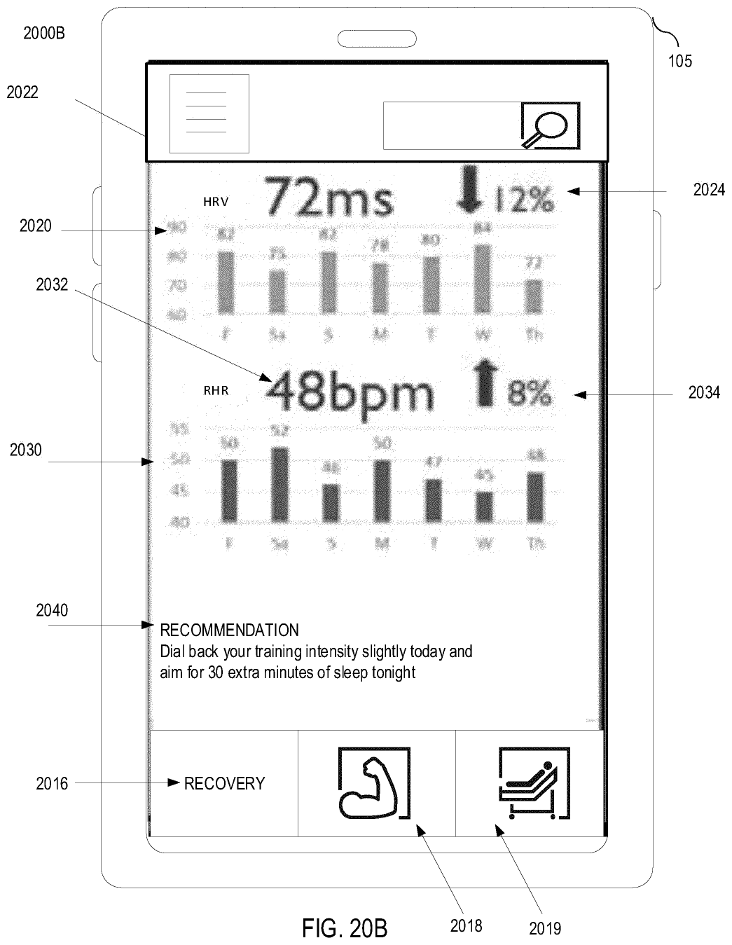

The MAHS and HPT application 157 may include a programming instructions for displaying human performance tracking (HPT) GUIs 162, as will be described in relation to FIGS. 20A and 20B. The system 100B can integrate a subject's monitored activities stored in the motion database 168 to provide a complete integrated solution for tracking human performance of a user and providing human performance data to a user using at least one graphical user interface (GUI) from the human performance tracking GUIs 162. In some embodiments, the monitored activity data may be provided by biometric sensor device 122, for example, as will be described in relation to FIG. 8.

The MAHS and HPT application 157 may include programming instructions for displaying standard of care (SoC) biometric GUIs 164 configured to display the biometric data of the standard of care biometric data including both the first set of biometric data and the second set of biometric data, as will be described in detail in relation to FIGS. 11, 12A, 13A, and 15, for example. The MAHS and HPT application 157 may include programming instructions configured to calculate and displaying health scores (HS) using HS GUIs 166.

FIG. 2 is a perspective view of a simplified representation of a biometric sensor device 200 as deployed for use on the skin 202 of a patient. The sensor device 200 is affixed to the skin 202 by way of an adhesive patch 204 that holds the sensor device 200 in position with its physiological characteristic sensor element 206 configured to be transcutaneously insertable into a biological system such as into the skin 202 of a subject or user. The sensor device 200 may be manufactured using waferscale fabrication technology on a common substrate with multiple sensor devices 200. The sensor device 200 includes the features, components, devices, and elements necessary to support both sensor-related functionality and wireless transmitter functionality. The wireless links 208 shown in FIG. 2 schematically illustrate that the sensor device 200 may be capable of supporting wireless data communication with one or more compatible devices, and without requiring another companion device or component connected thereto.

An example, biometric sensor device to sense basic metabolic panel biometric data is described in US Publication Application No. 2020/0072782, incorporated herein by reference as if set forth in full below. Other biometric sensor devices, such as for continuous glucose are described in US Publication Application Nos. US 2019/0090742 and 2019/009743 both of which are incorporated herein by reference as if set forth in full below.

FIGS. 3A-3D are examples of subsets of first modular set of biometric data. One or more of the subsets of the first modular set of biometric data may be captured or entered in a similar manner as the second modular set of biometric data. The various components of the first modular set of biometric data are configured to be stored in the biometric data database 159 and/or motion 168.

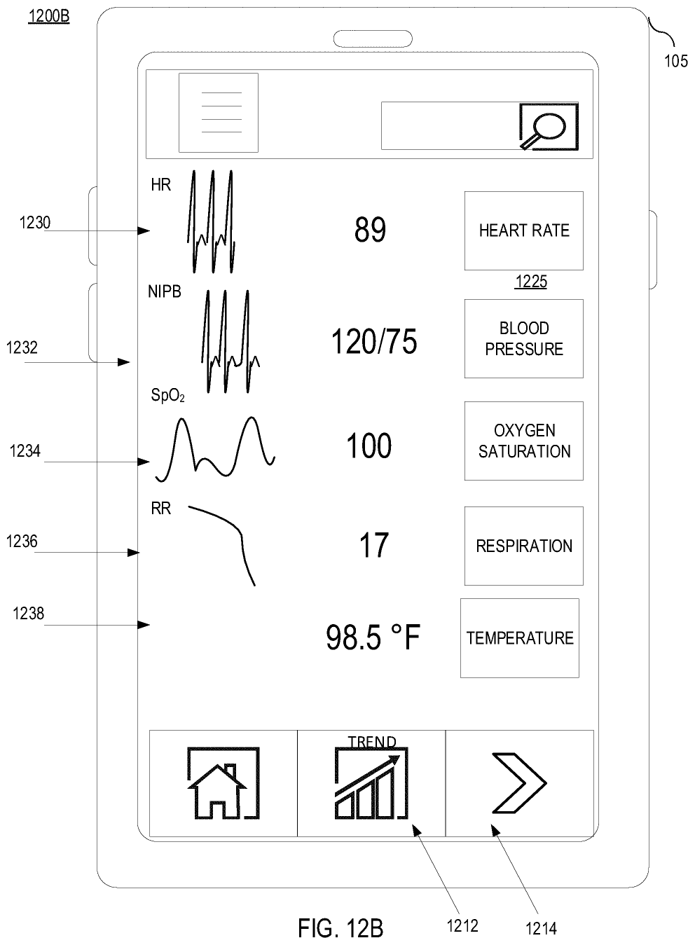

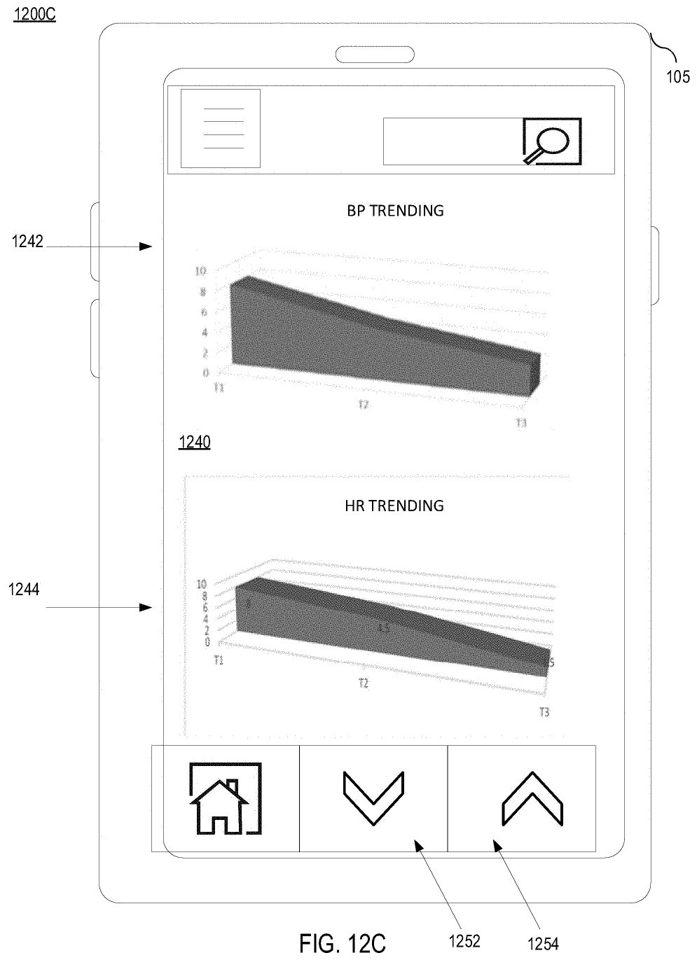

FIG. 3A is a block diagram of an embodiment of various components sensed by an implanted biometric sensor device 122. The biometric sensor device 122 configured to detect one or more cardiac function biometrics. For example, the cardiac function biometrics may include one or more sensors to sense blood pressure (BP) 302, heart rate (HR) 304, respiration rate (RR) 306, oxygen saturation (SpO₂) 308, and/or body temperature 310. The biometric sensor device 122 may include an inertial measurement unit (IMU) with an accelerometer, magnetometer and a gyroscope, for example, to detect activity or motion of the subject having the implanted continuous biometric sensor device 122. The activity or motion data 312 may be stored in the motion database 168. Any one or more of the biometrics illustrated may be individually selected, as shown in FIG. 12A where the application 157 includes instructions to cause the display of the continuously sensed biometric.

An example biometric sensor device is described in US Publication Application No. 2019/0336077 incorporated herein by reference as if set forth in full.

Sensors in the biometric sensor device 122 may include one or more of an inertial measurement unit; an electrocardiogram sensor; a photoplethysmogram; a thermometer; and/or a microphone.

FIG. 3B is a block diagram of an embodiment of various components generated by an implanted continuous infection detection sensor device 123. An implanted infection detection sensor device 123 may be configured to continuously sense one or more of lactate 322, pH 324, C-reactive protein (CRP) 326, and interleukin (IL-6) 328, for example Infection detection may also require parameters such as glucose. Biometrics for inflammation may include one or more of glucose, lactate, pH, CRP and IL-6. The pH sensor may include a voltammetric sensor. The lactate sensor may include an amperometric sensor. Measuring both lactate and pH simultaneously may be a leading biometric to detect infections before symptoms appear. The sensor device 123 may also sense glucose.

FIG. 3C is a block diagram of an embodiment of various components sensed by an implanted continuous basic metabolic panel sensor device 124. The implanted continuous basic metabolic panel sensor device 124 may include one or more sensors to sense the basic metabolic panel biometrics continuously. The sensor device 124 may sense sodium (NA) 332, chloride (CL) 334, blood bun 336, glucose (GLU) 338, creatinine (CR/SC) 340, bicarbonate/carbon dioxide 342 and/or potassium (K) 344. While, a continuous glucose sensor device may be provided as will be described in FIG. 3D, the modular framework of application 157 and system 150 may allow a user to use an implanted continuous glucose sensor device without the need for the continuous metabolic panel sensor device 124. In such an embodiment, the basic metabolic panel biometric data would be derived from a medical laboratory or hospital records, if an implanted sensor is not used for this subset of medical-grade biometrics.

FIG. 3D is a block diagram of an embodiment of a component generated by an implanted continuous glucose sensor device 350 is optional. The implanted sensor system 120 may also include an implanted continuous glucose sensor device 350. Alternately, the glucose biometric data may be derived by the implanted continuous basic metabolic panel sensor device 124.

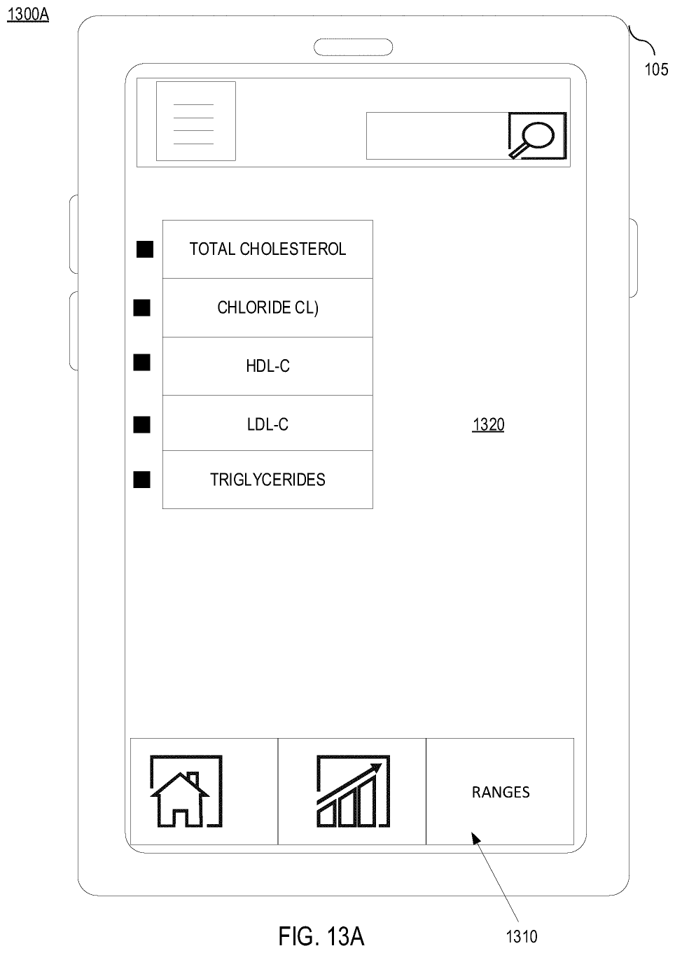

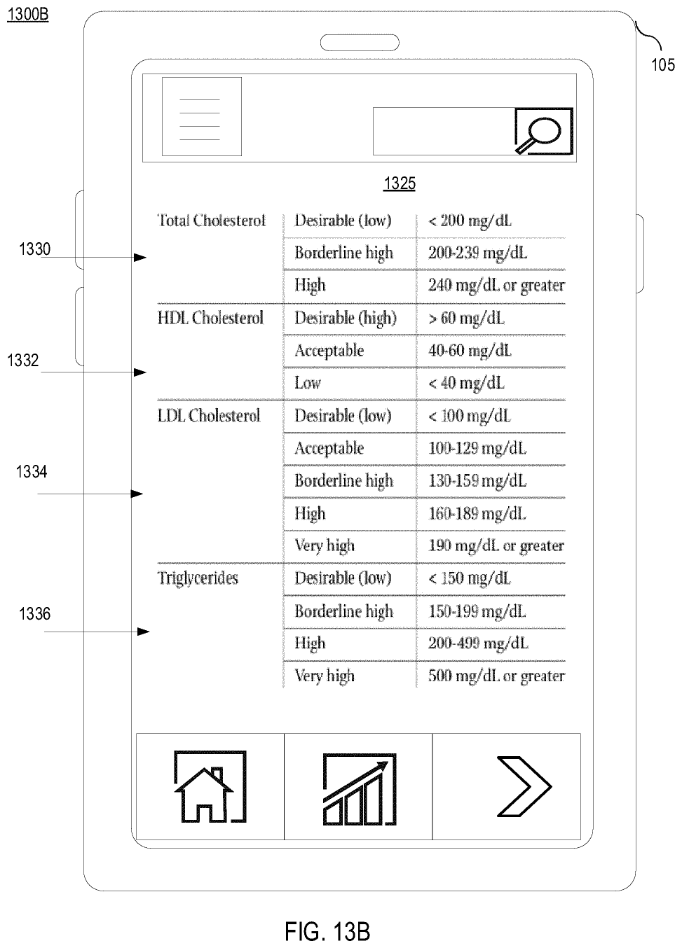

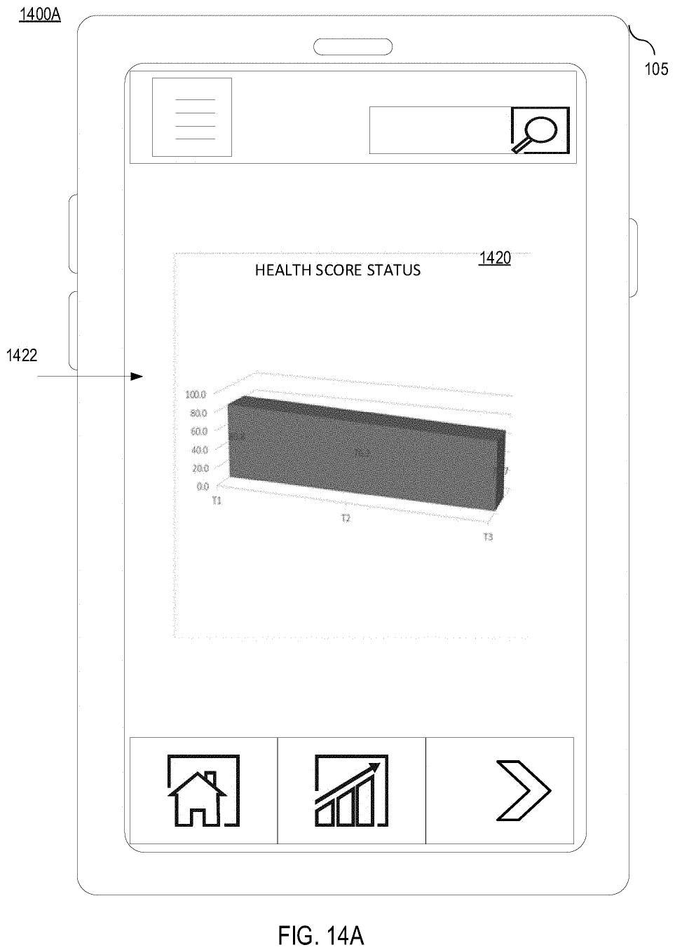

In addition to the sensed biometric data, the biometric data database 159 may also include range values for high, normal and low. Ranges may include ranges such as acceptable, borderline high, very high, for example. The ranges of the implanted sensor devices may be provided by the manufacturer of the different implanted sensor devices. The ranges may vary per manufacturer or medical laboratory. An example of range data for certain biometric data will be described in FIG. 13B. As can be appreciated, the ranges of the standard of care biometric data is known in the art.

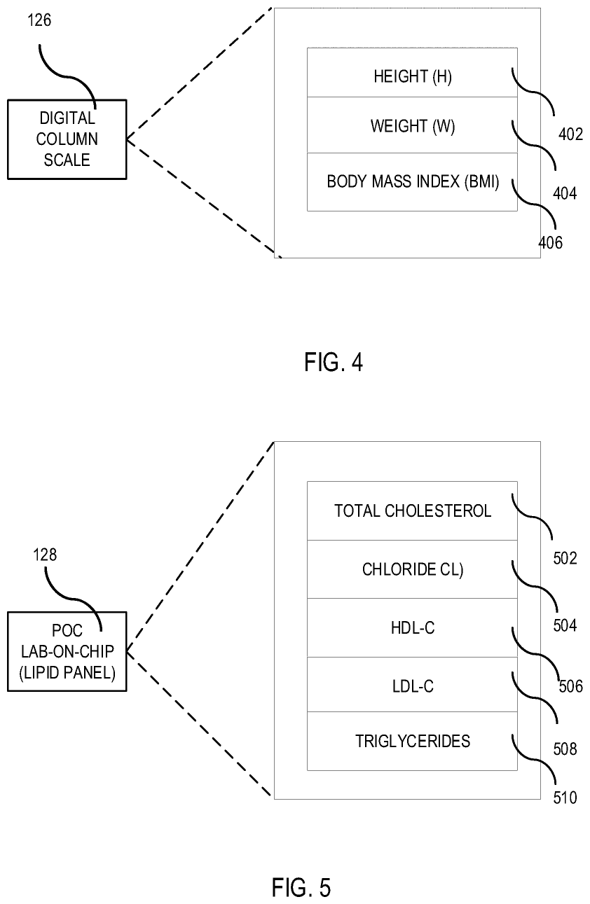

FIG. 4 is a block diagram of an embodiment of various components sensed by a digital column scale 126. The digital column sale 126 may sense height 402, weight 404 and/or body mass index 406. In some hospital facilities, the digital column scale 126 may communicate with electronic devices 101 using near field communications or BLUETOOTH wireless communications over a communication path.

FIG. 5 is a block diagram of an embodiment of various components sensed by a point of care (PoC) lab-on-chip device 128 associated with standard of care biometrics. The lab-on-chip device 128 may determine a lip panel that includes total cholesterol 502, chloride 504, high-density lipoprotein cholesterol (HDL-C) 506, low-density lipoprotein cholesterol (LDL-C) 508 and/or triglycerides 510.

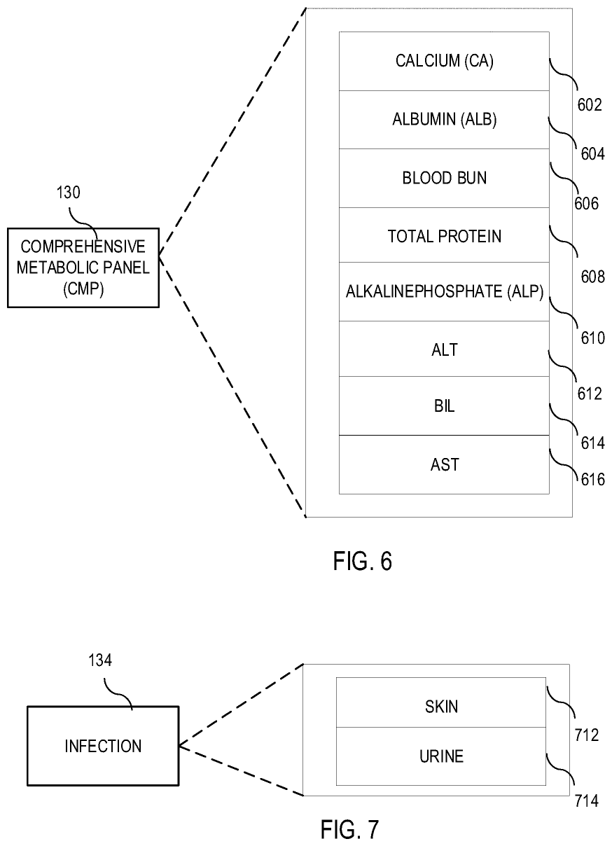

FIG. 6 is a block diagram of an embodiment of various components generated by a medical laboratory associated with standard of care biometrics. The medical laboratory during a hospital stay or for a physical of a subject may determine calcium 602, albumin 604, blood bun 606, total protein 608, alkalinephosphate 610, alanine aminotransferase (ALT) 612, basal insulin level (BIL) 614 and/or aspartate aminotransferase (AST) 616.

FIG. 7 is a block diagram of an embodiment of various components generated by a medical laboratory associated with standard of care biometrics. The standard of care biometrics may include information associated with other infection components. The infection 134 biometrics may include biometric data associated with a skin infection 712 and/or urine infection 714.

The medical-grade biometrics described above in relation to FIGS. 3A-3D, and 4-7 are for illustrative purposes and are not intended to be limiting in any way, as additional, biometrics may be used to determine a heath score of a user based on specific known illnesses they may have or previously had.

FIG. 8 is a block diagram of an embodiment of a user electronic device 105 interfacing with the modular ambulatory health status and performance tracking system 100B (FIG. 1B) to collect standard of care biometrics. The subject 10 is shown with the implanted sensor system 120. The implanted sensor system 120 may include the biometric sensor device 122 and a continuous basic metabolic panel sensor device 124. The implanted sensor system 120 may include a continuous infection detection sensor device 123.

The biometric sensor device 122 may include biometric sensors 802, activity senor 804 and a communication unit 806 to communicate with the user electronic device 105. The other sensor devices 123 and 124 may include its own communication unit 806 or share the communication unit 806 of sensor device 122. The modular biometric data 820 is described in FIGS. 3A-3D and may be stored in the biometric data database 159 (FIG. 1B) along with the ranges. The database 159 may be preloaded with ranges such as by entering sensor device identifiers.

The user electronic device 105 may communicate or receive data associated with a digital column scale 126, as shown in FIG. 4 that is stored in as digital column scale data 826 in the biometric data database 159 (FIG. 1B). The user electronic device 105 may communicate or receive data associated with lipid panel biometric data from a lab-on-chip 128, as shown in FIG. 5 that is stored as lipid panel data 828 in the biometric data database 159 (FIG. 1B) along with the ranges, for example.

The user electronic device 105 may communicate or receive data associated with comprehensive metabolic panel 130, as shown in FIG. 6 that is stored as comprehensive metabolic panel data 830 in the biometric data database 159 (FIG. 1B) along with the ranges. The user electronic device 105 may communicate or receive data associated with hematocrit 132 from a lab that is stored as hematocrit data 832 in the biometric data database 159 (FIG. 1B). The user electronic device 105 may communicate or receive data associated with infection 134, as shown in FIG. 7 that is stored as infection data 834 in the biometric data database 159 (FIG. 1B) along with the ranges. The collected standard of care biometrics are stored in memory for retrieval and analysis.

For example, if a subject was hospitalized for hypocalcemia which is a condition associated with low calcium, calcium may be monitored individually, as well as with the parameters associated with the standard of care biometrics. The doctor may provide other instructions (i.e., consult data 836) that may be stored in the biometric data database 159 (FIG. 1B). The doctor may request an alert using the doctor interface GUI 160 if one or more selected biometric data provides a specific health score, as will be described below, become out of a normal range or changes by a certain amount, for example

The user electronic device 105 may communicate or receive data associated with activity or motion data 868, based on sensed data by the activity sensor 804.

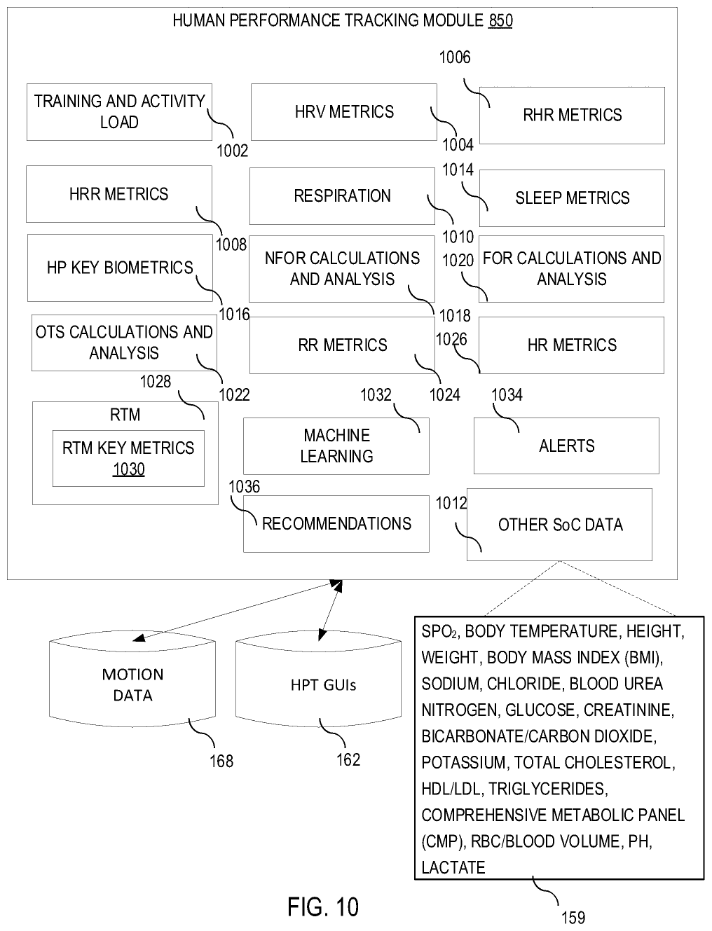

After the standard of care biometrics are stored, the data is analyzed by a health tracking module 840, as will be described in relation to FIG. 9. In some embodiments, after the standard of care biometrics are stored, the data may be analyzed by a human performance tracking module 850, as will be described in relation to FIG. 10. Since the system 100B is modular, the user may opt to only use the health tracking functionality of the system 100B. In other embodiments, the user may opt for only human performance tracking functionality of the system 100B. Still further, a user may opt for both the health tracking functionality and the human performance tracking functionality.

The sensor devices of the sensor system 120 are configured to provide continuous or nearly continuous sensor readings for use by one or both of health tracking module 840 and the human performance tracking module 850. In some cases, one or more biometrics of the standard of care biometrics may be updated periodically either manually or automatically in response to updated testing or physical evaluation.

The embodiments herein contemplate sharing any of the stored biometrics through an interface associated with a walk-in clinic, emergency room and medical facility, such as a hospital.

FIG. 9 is a block diagram of the health tracking module 840. The health tracking module 840 may include programming instructions, stored in memory 155, configured to allow a user to select biometrics using a biometric selector 920, as best seen in at least FIG. 11. The health tracking module 840 may include programming instructions configured to interface with biometric data 159, motion data 168 and/or HS GUIs 166, for example. The health tracking module 840 may include programming instructions configured to interface with other databases and instructions stored in memory 155.

The health tracking module 840 may include programming instructions configured to calculate health scores (HS) 950, as described below in relation to the Tables. The health tracking module 840 may include programming instructions configured to normalize biometric ranges 952. The health tracking module 840 may include programming instructions configured to calculate a health score trend 954 and/or a biometric trend 960. The health tracking module 840 may include programming instructions configured to generate an alert 970 in response to an out-of-range biometric, health score level or other setting. The health tracking module 840 may include programming instructions configured to display biometrics 972 including graphs and numerical values. The health tracking module 840 may include programming instructions for graphing trends 980 such as for health scores.

FIG. 10 is a block diagram of the human performance tracking module 850.

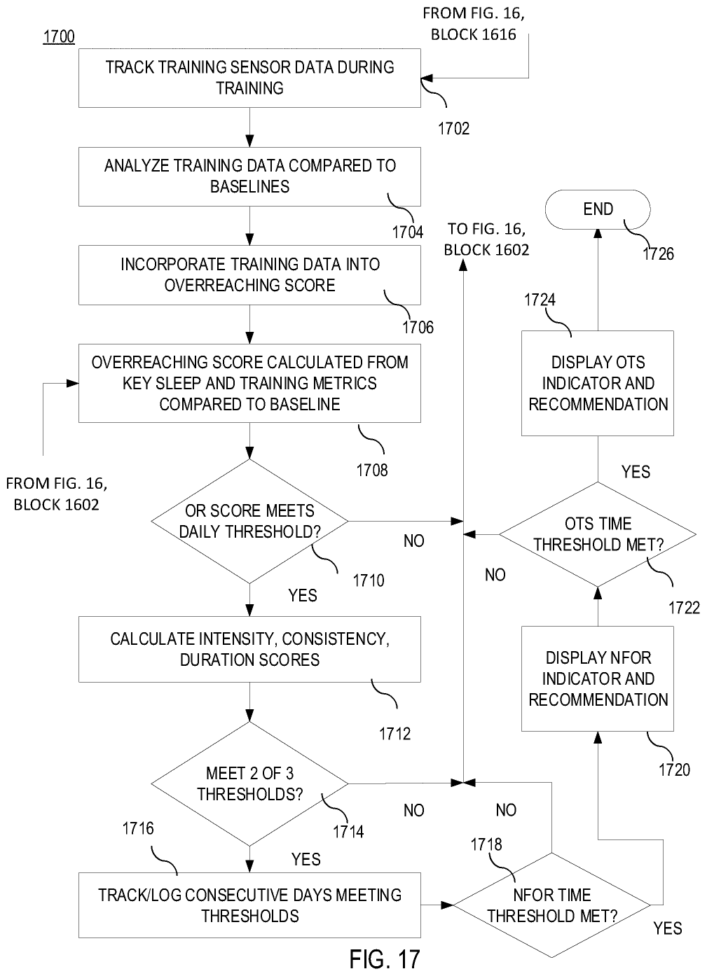

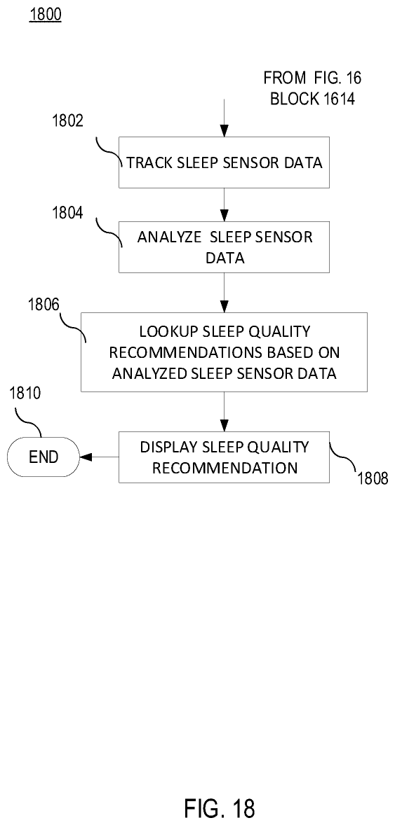

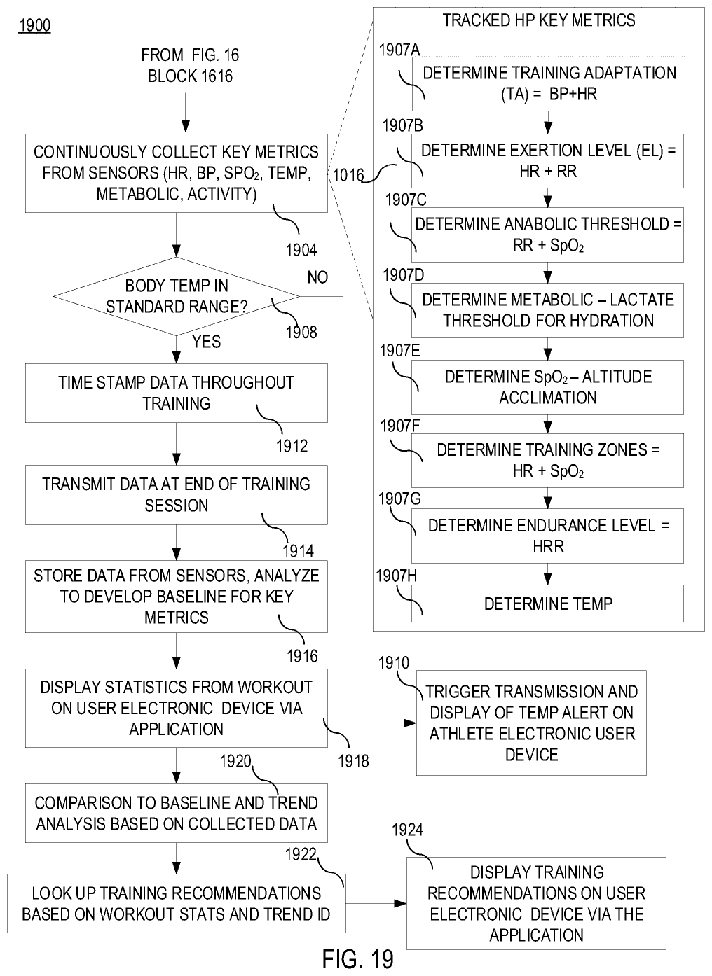

The human performance tracking module 850 may include programming instructions for analysis 170 that are configured to analyze training and activity load 1002, as described in FIGS. 16 and 17. The human performance tracking module 850 may configured to track rehabilitation activities, such as without limitation, those associated with physical therapy.

Various biometrics determined and evaluated by the human performance tracking module 850 are shown in Tables 6-9 below.

The human performance tracking module 850 may include programming instructions to calculate 172 that are configured to calculate and track one or more of heart rate variability (HRV) 1004, resting heart rate (RHR) 306, hear rate recovery (HRR) 1008, and respiration 1010. The human performance tracking module 850 may include programming instructions for analysis 170 that are configured to analyze other SoC biometric data 1012. The other SoC biometric data 1012 may be used to identify human performance key metrics, will be described in more detail in relation to FIGS. 16-19. Heart Rate Recovery is a measure of how quickly a subject's heart rate returns to normal rate immediately following exercise and is an indicator of both fitness and fatigue level. Movement/activity data will determine when an athlete is undertaking activity and when that activity ceases. At the point of activity termination, the algorithm will retrieve a HR measurement. Heart rate recovery is calculated by measuring the difference between HR during exercise and HR at time equals 1, 2 and 3 minutes immediately post exercise (HRR1, HRR2, and HRR3) being at three consecutive time stamps. The units of heart rate recovery are beats per minute (BPM).

Trends of increasing HRR indicate positive adaptation to training and increased cardiovascular conditioning. Short term decreased in HRR can indicate increased fatigue and decreased recovery status. Thus, thus high HRR (i.e., rapid recovery to normal HR) is indicative of high cardiovascular conditioning. Recognizing trends will be key for this utility. However, deviations of lower HRR from average can indicate high fatigue/low recovery from previous workout and should be factored into NFOR/OTS calculations. The cardiac parameters may include an HRV biometric. HRV biometric determines a change in the duration of each consecutive cardiac cycle. The cardiac cycle may be defined by the duration marked from the end of one heartbeat to the beginning of the next heartbeat. The cardiac cycle may include those phases corresponding to when the heart muscle relaxes and fills with blood or diastole phase; and a period of contraction and pumping of blood or systole phase. The HRV biometric may be measured by electrocardiogram (ECG) or via a pulse oximeter (photoplethsysmography or PTG), which may be part of the biometric sensor device 122. Predominant calculation used in research on HRV in athletes is log-transformed square root of the mean sum of the squared differences between R-R intervals. Resting/sleeping data should be used to avoid exertion leading to sympathetic interference. HRV measurements will be taken daily, while sleep and/or shortly after awakening. This is to avoid sympathetic interference due to activity or exertion. Single point calculations (i.e., daily value) will be displayed to user. Additionally, rolling 7-day and 30-day averages will be calculated. The system will identify trends in these averages and disregard inflections in daily value, as long-term positive or negative trends in HRV are more indicative of training adaptation and/or NFOR or OTS than daily levels.

The system may determine heart rate reserve, as will be described in TABLE 11.

The RHR biometric is the detected heart rate at rest. This measurement is tracked based on the motion data of the three-dimensional accelerometer (i.e., activity sensor 804) of the implanted biometric sensor 122, for example. The HRR biometric may be calculated based on the individual's performance and determine on a time based number (i.e., past week, past month, past 90 days, past year, etc.) by determining the actual HR max over the given time period and the HR rest over the same given time period. Example: if over the past 30 days, the heart rate (HR) max is 200 bpm and the HR rest (this can be a minimum number, or a rolling average say of the 5 minimum values) is 50 bpm, the HRR is 200−50=150. By way of non-limiting example, HR max may be determined as one of a peak number or a rolling average of the top 5 peak values over the past 30 days (or other period of time).

The human performance tracking module 850 may include programming instructions for analysis 170 that are configure to analyze sleep 1014 of a subject, as best described in at least FIG. 18. The human performance tracking module 850 may include programming instructions to calculate 172 that are configure to calculate or determine human performance (HP) key biometrics 1016, as best described in at least FIG. 19.

The HP key biometrics 1016 may include training adaptation (TA), exertion level (EL), anaerobic threshold, metabolic lactate threshold, SpO₂ an indicator for altitude acclimation, training zones, endurance level and body temperature. The calculations for these biometrics are described in FIG. 19 and described in detail in relation to TABLE 11.

The human performance tracking module 850 may include programming instructions to calculate 172 that are configured to calculate a nonfunctional overreaching (NFOR) metrics 1018; and programming instructions for analysis 170 that are configured to analyze the NFOR metrics 1018 to determine if a subject's training routine meets the NFOR metric 1018. The human performance tracking module 850 may include programming instructions to calculate 172 that are configured to calculate a functional overreaching (FOR) metrics 1020; and programming instructions for analysis 170 that are configured to analyze the FOR metrics 1020 to determine if a subject's training routine meets the FOR metric 1020. The human performance tracking module 850 may include programming instructions to calculate 172 that are configured to calculate an overtraining syndrome metric 1022; and programming instructions for analysis 170 that are configured to analyze the OTS metric 1022 to determine if a subject's training routine meets the OTS metric 1022.

The human performance tracking module 850 may include programming instructions to calculate 172 that are configured to calculate one or more of RR metrics 1024 and HR metrics 1026. The human performance tracking module 850 may include a recovery tracking module (RTM) 1028 with may include RTM key metrics 1030, as will be described in more detail below in relation to Table 6.

The human performance tracking module 850 may include programming instructions that execute machine learning algorithms 1032 which may be part of instructions for analysis 170 (FIG. 1B) that are configured to analyze the user's performance during training or rehabilitation of the physical body using motion. The performance of a subject includes determining trends associated with the training or rehabilitation activities. The memory 155 may include programming instructions using machine learning algorithms 1032 that when executed to cause a processor to analyze recovery of the monitored anatomy in response to training activities.

The human performance tracking module 850 may include programing instructions configured to interface with the biometric data database 159 the motion database 168 and the HPT GUIs 162, for example. The human performance tracking module 850 may interface with other databases and programming instruction stored in memory 155

The modularity of the MAHS and HPT application 157 (FIG. 1B) will be described in relation to FIG. 11. The illustrated GUIs are for the sake of illustration only and not meant to be limiting in any way.

FIG. 11 is a diagram of an embodiment of various components of a graphical user interface (GUI) 1100 for standard of care biometric navigation. Since some of the graphical user interfaces may use common navigation tools, the navigation tools of FIG. 11 will be described in detail. As other tools vary in other GUIs, those new tools will be described separately. The tools described in relation to any of the GUIs are for illustrative purposes and are not intended to be limiting in any way.

The GUI 1100 is configured to allow a user to selectively review standard of care biometrics or subset of biometrics. The subsets of biometrics are for illustrative purposes and not intended to be limiting in any way. The GUI 1100 may be configured to allow the user to download or manually enter one or more standard of care biometrics. The GUI 1100 may include a menu selection tab 1104 and a search field 1105 in tool bar 1102. The menu tab 1104 if selected may provide a drop down window of available functions. Other navigation tools may be used as well. The GUI 1100 may include a main GUI window 1120 configured to display the different subsets of standard of care biometrics. For example, the icon “cBS” if selected by the corresponding radio button 1121 or other selection tool, the programming instructions of the GUI 1100 may cause the GUI 1100 to switch to another GUI 1100 associated with the biometric data of the biometric sensor 122 (FIG. 1B), as will be described in more detail in relation to FIGS. 12A-12C.

The GUI 1100 may include additional navigation and selectin tools such as a home navigation selection button 1106, a graphs selection button 1108 and a patient file selection button 1110. The graphs selection button 1108 may navigate the user to continuously sensed data and health scores. The home navigation selection button 1106 may switch the current GUI 1100 to a home page (not shown). The graphs selection button 1108 may switch the continuous biometric data selection GUI in FIG. 12A. GUI 1100 may include icons to denote selection of other subsets of biometric data. By way of non-limiting example, the main GUI window 1120 may include icons for each subset of the second modular set of biometric data such as data received from a digital column scale 126 (FIG. 1B), a point of care (PoC) lab-on-chip device 128 (FIG. 1B) for providing biometric data associated with a lipid panel, a comprehensive metabolic panel 130 (FIG. 1B), hematocrit 132 (FIG. 1B) and certain infection data 134 (FIG. 1B). GUI 1100 may include icons for “Other” data such as for non-medical grade biometric data. For example, the modular framework may also allow user devices which sensor or monitor non-medical grade biometrics to be uploaded and stored in memory 155 (FIG. 1B) and selectively used for calculating a health score, as described below in relation to Table 5.

By way of non-limiting example, the home navigation selection button 1106 may allow the user to select a particular tracker, such as a standard of care tracker that is shown in FIG. 11 or a human performance tracker which is shown in FIGS. 20A-20B.

As will become evident from the description herein, the modularity may allow a user to only use data from an implanted sensor system 120. For example, after surgery a user may need to continuously monitor the biological system for signs of infection. In other cases, the user may only use data from the continuous biometric sensor 122 accompanied with a human performance tracker, as will be described in more detail in relation to FIGS. 16-19 and 20A-20B. Nonetheless, the modularity of the MAHS and HPT application 157 (FIG. 1B) may allow a user to monitor and track a suite of standard of care biometrics, as described herein, or selected subsets. Table 3 below is an example of a suite of standard of care biometrics used to determine a health score of a user. The health score is a function of the selected subsets of biometrics.

Still further, the MAHS and HPT application 157 (FIG. 1B) may include programming instructions that are configured to allow a user to selectively monitor their glucose for periodic reading daily.

Still further, the MAHS and HPT application 157 (FIG. 1B) may include programming instructions that are configured to selectively monitor a user's sleep patterns and activity, if an implanted biometric sensor device 122 is used, for example

The GUI 1100 may include programming instructions that allow a user to use the menu to import information or export information. The menu may allow the user to scan an image of the hardcopy of any of the standard of care biometric data and use feature extraction or optical character recognition (OCR) to recognize text of the hardcopy. Specifically, the numerical vales of the biometric data may be extracted and imported into the patient file, denoted by the patient file selection button 1110.

The list of menu functions is not exhaustive but only illustrative of example functionality

Assume for the sake of illustration, the radio button 1121 associated with the biometric sensor device 122 is selected. The selection of radio button 1121 is denoted by the black shading of the button 1121. After selecting radio button 1121 and selecting the graph selection button 1108, the programming instructions for GUI 1100 may cause the navigation to and display of GUI 1200 of FIG. 12A.

As can be appreciated, the icons and selection buttons are for illustrative purposes and not meant to be limiting in any way.

FIG. 12A is a diagram of an embodiment of various components of a GUI 1200A for identifying and/or selecting various components sensed by an implanted biometric sensor device 122. The GUI 1200A includes a main GUI window 1120 configured to display the selected biometric data entered via GUI 1100, for example. The GUI 1200A may also include a ranges selection button 1210 to navigate to the ranges of the selected biometrics selected via GUI 1200A. The GUI 1200A may include graph selection button 1208.

The selected biometric data may be from biometric sensor device 122 that senses blood pressure, heart rate, respiration rate, oxygen saturation, body temperature and/or activity, as previously described above in relation to FIG. 3A. Each of the biometrics of the biometric sensor device 122 may include a radio button 1222 for selection. A plurality of the biometrics are selected as denoted by the black radio button 1122. Assume now for the sake of illustrations that the user wants to see graphs and data of the continuous sensor biometric data. Thus, in an example, the user may select the graph selection button 1208. In response, the programming instructions of the GUI 1200A may cause the navigation to and display of GUI 1200B in FIG. 12B. Again, the graph selection button 1208 is just for illustrative purposes and other icons and buttons may be used based on the programming instructions.

FIG. 12B is a diagram of an embodiment of various components of a GUI 1200B for displaying in real-time biometrics of the various components sensed by an implanted biometric sensor device 122. The GUI 1200B includes a main GUI window 1225 configured to display real-time biometric data selected via GUI 1200A, for example. The main GUI window 1225 may include a graphical display of one or more sensed biometric data in a graph format over an increment of time associated including the current time. The main GUI window 1225 may include a graphical display of one or more sensed biometric data in text based format. Here, the main GUI window 1225 include various representation of the real-time data of heart rate 1230, blood pressure 1232, oxygen saturation 1234, respiration 1236 and body temperature 1238. If additional biometric data was selected and could not fit in the main GUI window 1225, a scroll button 1214 may be provided. In the illustration, the scroll button 1214 indicates the ability to scroll right.

The GUI 1200B may include a trending graph selection button 1212, for example Assume for the sake of illustration, the trending graph selection button 1212 was selected. Accordingly, the programming instructions for the GUI 1200B may cause the navigation to and the display of GUI 1200C of FIG. 12C.

FIG. 12C is a diagram of an embodiment of various components of a GUI 1200C for displaying a trending graph of various components of the implanted biometric sensor device. The GUI 1200C may include a main GUI window 1240 to display various trending graphs. The GUI 1200C may calculate and display a trending graph for each of the selected biometric data, selected in GUI 1200A. For the sake of illustration, a blood pressure trending graph 1242 and a heart rate trending graph 1244 are shown directly in the main GUI window 1240. Other trending graphs such as for the other selected biometrics associated with respiration rate, oxygen saturation, and body temperature are not shown but would also be accessible by scrolling up or down with a down scroll button 1252 and/or an up scroll button 1254 using the GUI 1200C. The calculations and algorithms for calculating and generating the blood pressure and heart rate trending graphs as described below in relation to Tables 1 and 2.

Returning again to FIG. 11, assume for the sake of illustration, the lipid panel biometric data was selected via GUI 1100 using the associated radio button. In such a case, the programming instructions of GUI 1100 may cause the navigation to and display of GUI 1300A.

FIG. 13A is a diagram of an embodiment of a GUI 1300A for identifying and/or selecting various components sensed by a point of care (PoC) lab-on-chip device associated with standard of care biometrics associated with a lipid panel. In the illustration, GUI 1200A includes a main GUI window 1320 which displays a list of biometric data associated with the lipid panel. The lipid panel may include total cholesterol, chloride, HDL-C, LDL-C and triglycerides, as previously described in FIG. 5. Assume for the sake of illustration, all of the biometric associated with the lipid panel are selected as denoted by the black shaded radio buttons. If the user selects a “Ranges” selection button 1310, the programming instructions of the GUI 1300A may cause the navigation to and display of GUI 1300B of FIG. 13B.

FIG. 13B is a diagram of an embodiment of a GUI 1300B for selectively identifying value ranges for various components sensed by a point of care (PoC) lab-on-chip device associated with standard of care biometrics of a lipid panel. In the illustration, GUI 1300B includes a main GUI window 1325 that displays the ranges at which each selected biometric date is high or low and any intermediate value ranges. Here, the total cholesterol may be displayed for low, borderline high and high. The HDL-C ranges 1332 may be displayed for high, acceptable and low. The LDL-C ranges 1334 may be displayed for low, acceptable, borderline high, high and very high. The triglycerides ranges 1336 may be displayed for low, borderline high and high and are displayed.

In the illustrated example, biometrics that were selected are displayed. However, if less biometrics are selected, then the display ranges may only include those selected or the arrangement of the display ranges may be arranged such that the selected ranges are displayed before non-selected ranges.

It should be noted that medical ranges of the standard of care biometric data are well established and vary per testing laboratory or manufacture of the sensors. The range of each biometric, as described below in relation to Tables 1-5, is used to normalize the numerical values of the biometric.

Although the examples of trending graphs are not shown, trending graphs of each biometric of the lipid panel may be determined over time as these biometrics are updated.

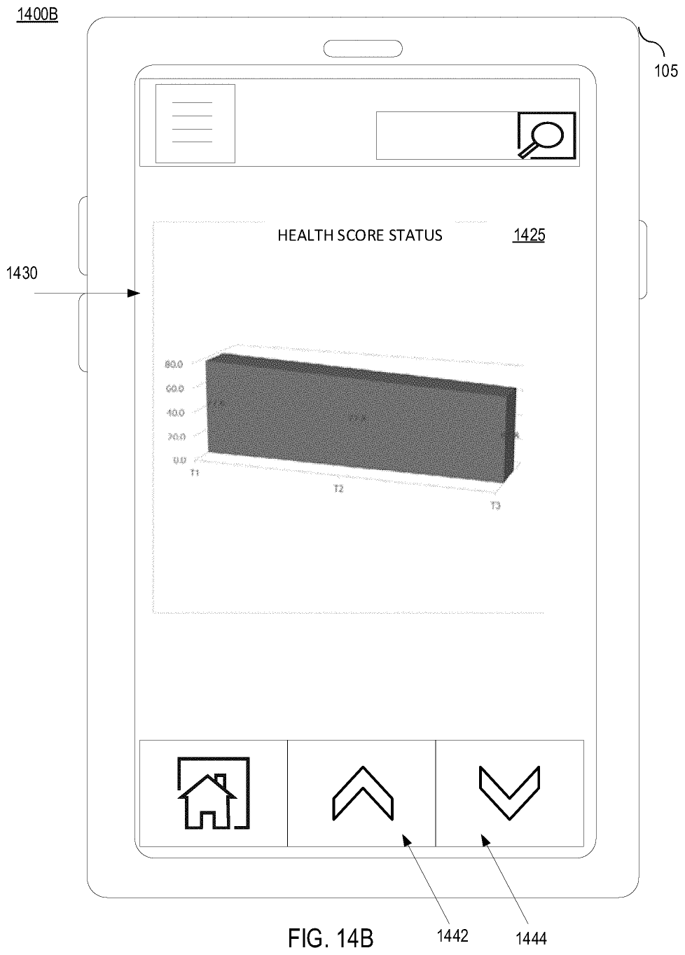

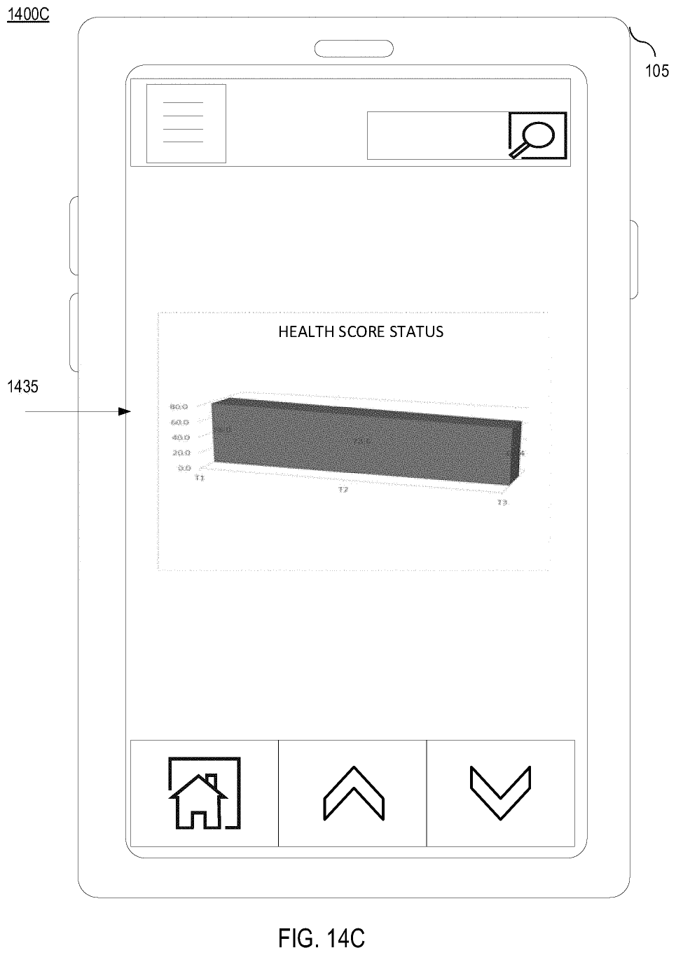

Returning again to FIG. 11, assume that using the navigational tools of the GUIs, the user selects to see a health score status. The health score status will be described in more detail in relation to FIGS. 14A-14C.

FIG. 14A is a diagram of an embodiment of a GUI 1400A for displaying a graph of a computed health score 1422 in a main GUI window 1420. In some embodiments, navigation to the graph of the health score may be display using the menu, the search field or other navigational tool. The health score 1422 is a graph of the user's health score over time. In this illustrations, the time periods are labeled T1, T2 and T3. While, this GUI 1400A provides a graph of the health score, a numerical value of the health score of an instantiation may be selectively displayed, although not shown. For example, times T1 and T2 may be in the past and the time T3 is the current time. Thus, the health score for time T3, is shown in one or more of the Tables below.

FIG. 14B is a diagram of an embodiment of a GUI 1400B for displaying a graph of a computed heath score 1430 in a main GUI window 1425. The GUI 1400B includes other navigation tools such as scroll up button 1442 and scroll down button 1444 to view other graphs. FIG. 14C is a diagram of an embodiment of a GUI 1400C for displaying a graph of another example of a computed health score 1435 using selected biometrics. The details of the calculations for generating the health score graphs in FIGS. 14A-14C are described below in relation to the discussion associated with Tables 3-5.

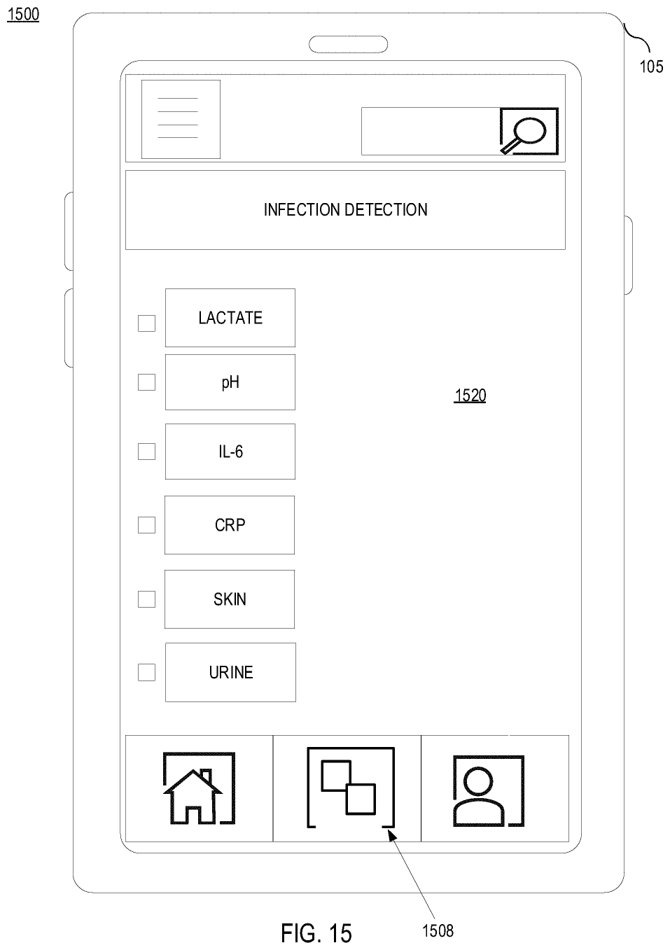

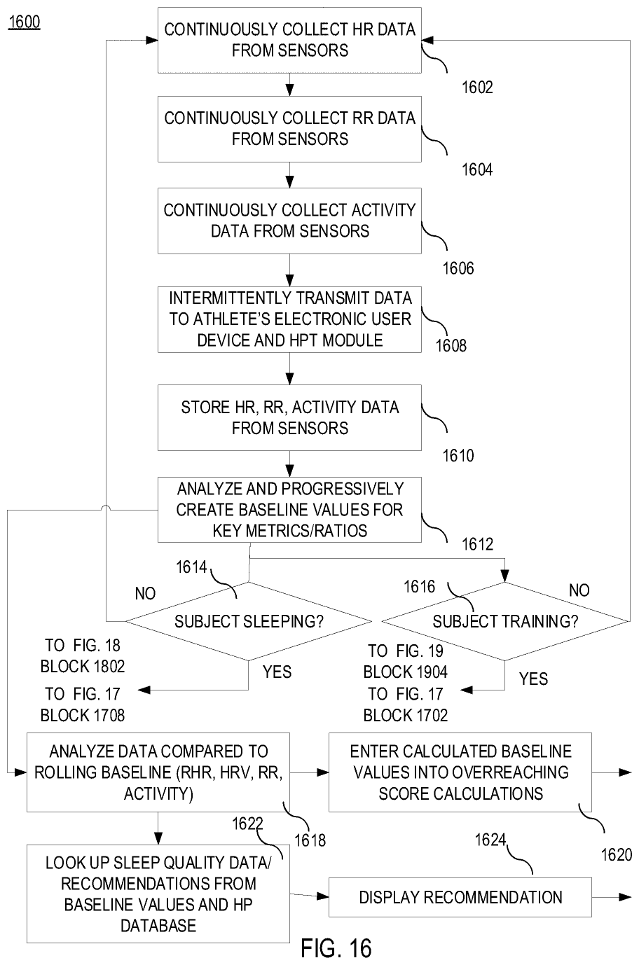

FIG. 15 is a diagram of an embodiment of a GUI 1500 for displaying infection biometric data. Infection biometric data may be received through different platforms as shown and described in relation to FIGS. 3B and 7. Returning again to FIG. 11, selecting the “cID” icon and “INFECTION” may cause the display of the infection biometric data of FIG. 15. The modularity of the MAHS and HPT application 157 (FIG. 1B) may include programming instructions that be configured to display in a main GUI window 1520 only the skin and urine biometrics, if the user does not have implanted a continuous infection detection sensor device 123. In other cases, the lactate, pH, IL-6 and CRP biometrics may be displayed in addition to the biometrics for skin and urine infections, if the continuous infection detection sensor device 123 is implanted. In the illustration, the navigation tools includes data archive button 1508.

Each of the available biometrics associated with infection biometric data is individually selectable via a radio button or other selection tool.

### Standard of Care Tracker

### Health Score

In an embodiment, the health tracking module 840 is configured to calculate a relative sickness/wellness (or health status) score that is normalized on a scale of 0 to 100, as shown in FIG. 14A, based on the integration of a suite of 30 standard of care biometrics, for example. A sample integration algorithm and method for trending a patient's relative sickness/wellness over time is described below. The health tracking module 840 may also determine a health score for any one particular biometric of the suite of standard of care biometrics. Each sensed biometric is displayed as a score of a biological system of the subject. A glucose reading is a score. Blood pressure is a score. Each score may be normalized individually or collectively to generate comprehensive score.

### Trending Graph (Biometric)

In general, each standard of care biometric may be a defined “normal value” with acceptable upper/lower limits. Beyond these acceptable upper/lower limits is a band of “medical concern” values and beyond that is a band of “medical crisis” values.

Referring again to FIG. 12C, the trending graphs 1242 and 1244 for various components of the implanted biometric sensor device 123 will now be described. The trending graph 1242 is for blood pressure. The blood pressure (BP) biometric 302 (FIG. 3A) may have an acceptable normal range of 120+/−9 mmHg for systolic pressure and 80+/−9 mmHg for diastolic pressure. A band of “medical concern” values between 130/90 and 180/120 mmHg for HIGH BP and 109/69 and 90/60 mmHg for LOW BP. The level of blood pressure may include a band for “medical crisis” values above 180/120 mmHg for HIGH BP and below 90/60 mmHg for LOW BP. It should be noted that these ranges may be programmatically changed or updated such as when medical guidelines change. For example, in 2018 the American College of Cardiology (ACC) and American Heart Association (AHA) adopted new guidelines that defines elevated/high blood pressure as any readings above 120/80 mmHg. In some embodiments, a doctor or healthcare practitioner may alter the levels as necessary.

### Example Trending Graph: Blood Pressure

In an embodiment, the results of the blood pressure 302 (FIG. 3) can be normalized on a scale of 0 to 10 with 10 being defined as “ideal”. The term “ideal” related to biometric ranges is based on ranges for a population that may vary based on age, race, sex and geographical location. For a blood pressure 302 of 131/90 mmHg at time T1, a normalized health scale result of “8” can be assigned. For a blood pressure result of 155/102 at time T2, a normalized health scale result of “5” can be assigned. For a blood pressure result of 160/110 at time T3, a normalized health scale result of “3” can be assigned. In Table 1, an example normalized scale for the biometric parameter associated with blood pressure is shown.

TABLE 1

| Normalized Scale | HIGH BP | LOW BP | Alert |
|---|---|---|---|
| 10 | 120/80 | 120/80 | “ideal” range |
| 9 | 129/89 | 110/70 | |
| 8 | 134/92 | 107/68 | medical |
| 7 | 139/95 | 104/67 | concern |
| 6 | 144/97 | 102/66 | |
| 5 | 150/100 | 100/65 | |
| 4 | 157/105 | 98/64 | |
| 3 | 165/110 | 95/63 | |
| 2 | 172/115 | 93/61 | |
| 1 | 180/120 | 90/60 | |
| 0 | above 180/120 | below 90/60 | medical crisis |

Accordingly, the MAHS and HPT application 157 (FIG. 1B) may include programming instructions for generating an alert based on a “medical concern” value or a “medical crisis.” In some embodiments, the doctor may have entered instructions to provide an alert to a contact number, email, or other designated contact information if a “medical crisis” is detected by MAHS and HPT application 157.

As another example, if blood pressure trending can be shown for T1=“8”, T2=“5”, T3=“3” indicating clearly that this patient's BP results are trending away from the “ideal” and cause for medical concern (but not a medical crisis). The time interval for determining and graphing a “trend” is user selectable by increments of 5 minutes, hourly, 4 times a day, etc., using the MAHS and HPT application 157. Hence, a trend (i.e., amount of change) can be used as a basis of generating an alert and not just a raw number at any instantiation in time.

### Example Trending Graph: Heart Rate

Using heart rate 304 (FIG. 3A), as an example biometric, the heart rate (HR) 304 may have an acceptable normal range of 60 to 100 beats per minute at rest. A band of “medical concern” values between 101 and 250 bpm for HIGH HR may be representative of tachycardia. Heart rate values between 59 and 30 bpm for LOW HR may be representative of bradycardia. Additionally, a band of “medical crisis” values above 250 bpm for HIGH HR and below 30 bpm for LOW HR may be set as a basis of alert. These values and normal ranges are subject to change as medical guidelines change. Furthermore, alerts may be a function of the user's own physiology and biology. As an example, highly trained athletes can have resting heart rates between 30 and 60 bpm without “medical concern” as their cardiac output has greater efficiency than the average person.

Therefore, results of a heart rate 304 may be normalized on a scale of 0 to 10 with 10 being defined as “ideal”. For a heart rate result of 122 beats per minute (bpm) at time T1, a normalized health scale result of “8” can be assigned. For a heart rate result of 185 at time T2, a normalized health scale result of “4.5” can be assigned. For a heart rate result of 228 at time T3, a normalized health scale result of “1.5” can be assigned, such as shown in Table 2.

TABLE 2

| Normalized Scale | HIGH HR | LOW HR | Alert |
|---|---|---|---|
| 10 | 80 bpm | 80 bpm | “ideal” range |
| 9 | 100 bpm | 60 bpm | |
| 8 | 119 bpm | 57 bpm | medical |
| 7 | 137 bpm | 53 bpm | concern |
| 6 | 156 bpm | 49 bpm | |
| 5 | 175 bpm | 45 bpm | |
| 4 | 190 bpm | 42 bpm | |
| 3 | 205 bpm | 39 bpm | |
| 2 | 220 bpm | 36 bpm | |
| 1 | 235 bpm | 33 bpm | |
| 0 | above | below | medical crisis |

In another example, heart rate trending can be shown for T1=“8”, T2=“4.5”, and T3=“1.5” indicating clearly that this user's heart rate is trending away from the “ideal” and cause for medical concern (but not a medical crisis). The time interval for which a “trend” is determined and graphed for any one biometric is selectable by increments of 5 minutes, hourly, 4 times a day, etc., using the MAHS and HPT application 157 (FIG. 1B).

Each biometric of the suite of standard of care biometrics will be normalized in a manner similar to what is described above. For example, the ranges for a user's lipid panel is received from a laboratory, for example, as shown in FIG. 13B. The MAHS and HPT application 157 may be configured to normalize the ranges from the laboratory in some examples or use a normalized scale based on standardized ranges for the same biometric.

### Example Trending Graph: Modular Suite of Standard of Care Biometrics

### Example 1

The health score using the suite of standard of care biometrics will now be described. Table 3 represents a selected suite of the standard of care biometrics after being normalized for times T1, T2 and T3. The selected suite of the standard of care biometrics may include those biometrics described in FIGS. 3A, 3C-3D, and FIGS. 4-7.

TABLE 3

| | Biometric | T1 | T2 | T3 |
|---|---|---|---|---|
| 1 | blood pressure (BP) | 8 | 5 | 3 |
| 2 | heart rate (HR) | 8 | 4.5 | 1.5 |
| 3 | respiration rate (RR) | 9 | 7.5 | 6 |
| 4 | oxygen saturation (SpO₂) | 7.5 | 7 | 6.5 |
| 5 | temperature (TEMP) | 10 | 10 | 9.5 |
| 6 | height H (always normalizes to 10) | 10 | 10 | 10 |
| 7 | weight W (relative to height) | 7 | 7 | 7 |
| 8 | body mass index (BMI) | 7.5 | 7.5 | 7.5 |
| 9 | BMP - sodium (Na) | 8 | 7 | 6 |
| 10 | BMP - chloride (Cl) | 7 | 7 | 7.5 |
| 11 | BMP - blood urea nitrogen (BUN) | 10 | 9.5 | 9.5 |
| 12 | BMP - glucose (Glu) | 9 | 9 | 8.5 |
| 13 | BMP - Creatinine (CR/Scr) | 7 | 6.5 | 6 |
| 14 | BMP - bicarbonate/carbon dioxide | 8 | 7 | 6.5 |
| 15 | BMP - potassium (K) | 7 | 6.5 | 5.5 |
| 16 | total cholesterol | 8 | 8 | 8 |
| 17 | high-density lipoprotein | 8 | 8 | 8 |
| 18 | low-density lipoprotein cholesterol | 6 | 6 | 6 |
| 19 | Triglycerides | 7 | 7 | 7 |
| 20 | CMP - calcium (Ca) | 9 | 9 | 8 |
| 21 | CMP - albumin (ALB) | 7 | 6 | 5 |
| 22 | CMP - total protein (TP) | 9 | 9 | 9 |
| 23 | CMP - alkaline phosphatase (ALP) | 10 | 9 | 10 |
| 24 | CMP - alanine transaminase (ALT) | 10 | 10 | 9 |
| 25 | CMP - aspartate aminotransferase (AST) | 10 | 10 | 10 |
| 26 | CMP - bilirubin (BIL) | 9 | 8 | 8 |
| 27 | Hematocrit | 9.5 | 9.5 | 9.5 |
| 28 | skin - bacteria culturing | 10 | 10 | 10 |
| 29 | urine - bacteria culturing | 2 | 2 | 2 |
| 30 | patient/physician consult (1-10 scale) | 5 | 5 | 5 |
| | TOTAL Health Score (0-300) scale | 242.5 | 227.5 | 215.0 |
| | NORMALIZED Health Score (0-100 scale) | 80.8 | 75.8 | 71.7 |

The algorithm and methodology applied to Tables 1 and 2 have been completed for the remaining 28 biometrics, as shown in Table 3. The normalized biometrics may be used to obtain a cumulative health score numerator value with a denominator of 300. In other words, the sum of the medical-grade 30 normalized biometric results are divided by 300 (i.e., 30 biometrics×10 for maximum normalized scale) to obtain a normalized aggregate health score on a scale of 0 to 100 with 100 being the “ideal” result. Again, the aggregated health score results can be plotted over time to show a general patient trend towards sickness (trending towards 0) or wellness (trending towards 100), as best seen in FIG. 14A. For this example, T1=80.8, T2=75.8, and T3=71.7. The trending graph 1422 in FIG. 14A may represent a trend indicative of the user's health trending towards sickness (or simply stated, the user's condition is getting worse).

### Example 2

Table 4 is an example of a selected suite of standard of care biometrics with a portion of the biometrics shown in Table 3 removed.

TABLE 4

| | Biometric | T1 | T2 | T3 |
|---|---|---|---|---|
| 1 | blood pressure (BP) | 8 | 5 | 3 |
| 2 | heart rate (HR) | 8 | 4.5 | 1.5 |
| 3 | respiration rate (RR) | 9 | 7.5 | 6 |
| 4 | oxygen saturation (SpO₂) | 7.5 | 7 | 6.5 |
| 5 | temperature (TEMP) | 10 | 10 | 9.5 |
| 6 | height H (always normalizes to 10) | 10 | 10 | 10 |
| 7 | weight W (relative to height) | 7 | 7 | 7 |
| 8 | body mass index (BMI) | 7.5 | 7.5 | 7.5 |
| 9 | BMP - sodium (Na) | 8 | 7 | 6 |
| 10 | BMP - chloride (Cl) | 7 | 7 | 7.5 |
| 11 | BMP - blood urea nitrogen (BUN) | 10 | 9.5 | 9.5 |
| 12 | BMP - glucose (Glu) | 9 | 9 | 8.5 |
| 13 | BMP - Creatinine (CR/Scr) | 7 | 6.5 | 6 |
| 14 | BMP - bicarbonate/carbon dioxide | 8 | 7 | 6.5 |
| 15 | BMP - potassium (K) | 7 | 6.5 | 5.5 |
| 16 | total cholesterol | 8 | 8 | 8 |
| 17 | high-density lipoprotein cholesterol (HDL-C) | 8 | 8 | 8 |
| 18 | low-density lipoprotein cholesterol (LDL-C) | 6 | 6 | 6 |
| 19 | Triglycerides | 7 | 7 | 7 |
| 20 | Hematocrit | 9.5 | 9.5 | 9.5 |
| 21 | skin - bacteria culturing | 10 | 10 | 10 |
| 22 | urine - bacteria culturing | 2 | 2 | 2 |
| 23 | patient/physician consult (1-10 scale) | 5 | 5 | 5 |
| | TOTAL Health Score (0-230) scale | 178.5 | 166.5 | 156.0 |
| | NORMALIZED Health Score (0-100 scale) | 77.6 | 72.8 | 67.8 |

In FIG. 14B, a health score trending graph 1430 is shown with a scale from 0 to 100. The graph ends at 80 since the scores are below 80. The MAHS and HPT application 157 may include programming instructions configured to determine a modular health score based on selected biometrics or those biometrics available. By way of non-limiting example, the modular health score may be adjusted by data normalization. For instance, the comprehensive metabolic panel (CMP) may be used to evaluate liver function and could be evaluated in a separate health scoring. In the example of Table 4, a health score using only a subset of standard of care biometrics is determined by eliminating the comprehensive metabolic panel. Here, a suite of 23 standard of care biometrics have been user selected or selected by the application 157. The health score is normalized by dividing the total health score by 230 (i.e., 23 biometrics×10 for the normalized scale). The health score results include for time T1=77.6, T2=72.8 and T3=67.8. Again, the example the user's health score is worsening. The health score trend graph is representative of a trend toward sickness.

### Example 3

Another example of a health score uses additional biometric sensors inputs that can be added to the MAHS and HPT application 157 and system 100B by adjusting the data normalization routine. For instance, an activity tracker such as one that is incorporated into a smart watch 107 or other activity tracker, such as FITBIT ONE, may capture and evaluate daily steps and sleep quality. The smart watch 107 and activity tracker are not medical grade devices. However, biometric data from these non-medical grade sensors could be added to the medical grade suite of standard of care biometrics. In this example, the smart watch 107 or activity tracker may be configured to communicate with the user electronic device 101 (i.e., smart phone 105) to transfer data to the application 157 for subsequent transfer to the biometric database 159. An example of biometric data with added non-medical grade biometric data is shown in Table 5.

TABLE 5

| | Biometric | T1 | T2 | T3 |
|---|---|---|---|---|
| 1 | blood pressure (BP) | 8 | 5 | 3 |
| 2 | heart rate (HR) | 8 | 5.5 | 1.5 |
| 3 | respiration rate (RR) | 9 | 7.5 | 6 |
| 4 | oxygen saturation (SpO₂) | 7. | 7 | 6.5 |
| 5 | temperature (TEMP) | 10 | 10 | 9.5 |
| 6 | height H (always normalizes to 10) | 10 | 10 | 10 |
| 7 | weight W (relative to height) | 7 | 7 | 7 |
| 8 | body mass index (BMI) | 7.5 | 7.5 | 7.5 |
| 9 | BMP - sodium (Na) | 8 | 7 | 6 |
| 10 | BMP - chloride (Cl) | 7 | 7 | 7.5 |
| 11 | BMP - blood urea nitrogen | 10 | 9.5 | 9.5 |
| 12 | BMP - glucose (Glu) | 9 | 9 | 8.5 |
| 13 | BMP - Creatinine (CR/Scr) | 7 | 6.5 | 6 |
| 14 | BMP - bicarbonate/carbon | 8 | 7 | 6.5 |
| 15 | BMP - potassium (K) | 7 | 6.5 | 5.5 |
| 16 | total cholesterol | 8 | 8 | 8 |
| 17 | high-density lipoprotein cholesterol (HDL-C) | 8 | 8 | 8 |
| 18 | low-density lipoprotein cholesterol (LDL-C) | 6 | 6 | 6 |
| 19 | Triglycerides | 7 | 7 | 7 |
| 20 | CMP - calcium (Ca) | 9 | 9 | 8 |
| 21 | CMP - albumin (ALB) | 7 | 6 | 5 |
| 22 | CMP - total protein (TP) | 9 | 9 | 9 |
| 23 | CMP - alkaline phosphatase (ALP) | 10 | 9 | 10 |
| 24 | CMP - alanine transaminase (ALT) | 10 | 10 | 9 |
| 25 | CMP - aspartate aminotransferse (AST) | 10 | 10 | 10 |
| 26 | CMP - bilirubin (BIL) | 9 | 8 | 8 |
| 27 | Hematocrit | 9.5 | 9.5 | 9.5 |
| 28 | skin - bacteria culturing | 10 | 10 | 10 |
| 29 | urine - bacteria culturing | 2 | 2 | 2 |
| 30 | patient/physician consult (1-10 scale) | 5 | 5 | 5 |
| 31 | daily steps (7K minimum, 15K ideal) | 2 | 2 | 2 |
| 32 | sleep quality (1-10 scale) | 5 | 5 | 5 |
| | TOTAL Health Score (0-320 scale) | 249.5 | 235.5 | 222.0 |
| | NORMALIZED Health Score (0-100 scale) | 78.0 | 73.6 | 69.4 |

In Table 5, the biometrics 31 and 32 have been added from a non-medical grade sensor device, such as an activity tracker. For this example, with 32 biometrics, the health score is at time T1=78.0 as compared to 80.8 for 30 biometrics. The health score is at time T2=73.6 as compared to 76.2 for 30 biometrics. The health score is at time T3=69.4 as compared to 71.7 for 30 biometrics. Again, the health scores in the graph 1435 of FIG. 14C shows the user's health status is worsening or trending towards sickness. The term “K” is equal to 1000.

Adding a consumer device such as a FITBIT ONE® into the medical-grade biometrics stored in biometric data database 159 to measure steps and sleep quality does not meet the medical-grade standard of care criteria for making medical claims. The measurement of “steps” is not an accepted medical criterion, nor is sleep quality determined by movement only during sleep. The point being that adding additional biometric data into the system 150 still reinforces the overall health status trending.

### Example 4

Returning to Table 4, assume for the sake of illustration, another modular medical-grade biometric sensor data or test data is available. In this example, assume Hemoglobin A1C, Prothrombin (PT), etc., is available to the user and system 150 and can be modularly added to via the MAHS and HPT application 157 to the biometric data database 159 (FIG. 1B). The GUI 1100 in FIG. 11 may be updated with an indication (not shown) of additional modular biometric data. The additional modular data may be used for selectively developing health scores using medical-grade biometric data for tracking conditions associated with certain health conditions.

### Human Performance Tracker

By comprehensively studying the performance of these model systems from the molecular level to macroscopic biomechanics, the effects of training and conditioning on their physiology, and the ability to optimize well beyond their ‘set point’, we can gain insight into how to engineer health and wellness. Insights can be gained into understanding and detecting early signs of neuromotor fatigue in order to prevent impending systematic failure. Such findings may, for example, better inform athletes or users on how to prevent injuries, combat stress and obesity as well as improve recovery and rehabilitation.

### Overreaching and Overtraining

An athlete may, for example, typically implement some form of “periodization” into their training program. Physical stress and exertion can temporarily diminish an athlete's physical ability in what is called “functional overreaching”, a desired state. When executed properly, the body's adaptation to this stress may subsequently recover the athlete's physical ability to a level higher than it was prior to the stress in a process called “supercompensation”. This process may require a precise balance of training load and recovery. An imbalance of these factors can lead to undertraining and a lack of progress or nonfunctional overreaching and further to possibly overtraining syndrome.

The embodiments herein track three conditions (functional overreaching, nonfunctional overreaching and overtraining syndrome) on a spectrum. Functional overreaching is a desired state with short-term physical deficits, such as weakness, fatigue and lack of endurance. Nonfunctional overreaching results from either a higher training/recovery imbalance or that imbalance being sustained for a longer period. The same symptoms may likely present, but more intense and longer time to recover. The athlete may also likely not achieve the same desired supercompensation. When nonfunctional overreaching is sustained for long periods, it can turn into overtraining syndrome. Hence, the embodiments herein may detect early signs of overtraining syndrome. Again, similar symptoms may be present, but much more severe and much longer to recover (if at all). The primary differentiator between functional overreaching, nonfunctional overreaching and overtraining syndrome is the (1) intensity (2) consistency and (3) duration of symptoms.

The challenge for an athlete or a coach is to strike the appropriate balance of training and recovery to achieve functional overreaching and supercompensation while avoiding nonfunctional overreaching and the overtraining syndrome. This is currently a challenge for athletes because it is a highly subjective measure; different for each individualized athlete physiology; and not precisely “measurable” with existing technology and biometrics.

### Recovery Tracking Module

A goal of the recovery tracking module 1028 (FIG. 10) may include programming instruction configured to provide the athlete with an objective measure of their recovery status to accurately identify the thresholds of functional overreaching, nonfunctional overreaching and overtraining syndrome and provide recommendations for optimal training and recovery. This may be accomplished by establishing baseline levels and monitoring deviations from baseline values for six RTM key biometrics 1030. The six RTM key biometrics 1030 may include: 1) Resting Heart Rate (RHR) during sleep; 2) Heart Rate Variability (HRV) during sleep; 3) Sleep disturbances; 4) Exertion level during activity; 5) Heart Rate Recovery (HRR) after exertion; and 6) Heart Rate (HR) during exertion. The RTM biometrics may also include Heart Rate Reserves,

Implantable sensor system 120 may constantly collect data on the following biometrics, as shown in Table 6:

TABLE 6

| Biometric | Detail |
|---|---|
| R-R intervals | Sensed electrically, filtered and cleansed using signal |
| | processing and ectopy rejection algorithm |
| Activity Level | Utilized three-dimensional accelerometer |
| Respiration | Combination of accelerometer and R-R sensing; Measure |
| | of exertion due to strong correlation to Rated Perceived |
| | Exertion (RPE) |

The RPE data may be determined based on a Borg RPE scale. The Borg RPE scale may be a numerical scale that ranges from 6 to 20, where 6 means “no exertion at all” and 20 means “maximal exertion.” When a measurement is taken, a number is chosen from the Borg RPE scale stored in memory. [“Perceived Exertion (Borg Rating of Perceived Exertion Scale”, www.cdc.aov/physicalactivity/basics/measuring/exertion)] The score may describe a subject's level of exertion during physical activity. A value of 6 on the same may represent a no exertion level. A value of 7 may represent a level of extremely light exertion. A value of 9 may represent a level of very light exertion. A value of 11 may represent a light exertion level. A value of 23 may represent a somewhat hard exertion level. A value of 15 may represent a hard exertion level. A value of 17 may represent a very hard exertion level. A value of 19 may represent an extremely hard exertion level. A value of 20 may represent a maximum exertion level.

The RPE score×10=HR. For example, RPE score of 15 indicates a HR=150 bpm. The RPE may be subjective and self-reported, and an objective measure of exertion will allow for more accurate analysis of performance adaptation.

Data on these biometrics in Table 6 may be transmitted via wireless communications, such as BLUETOOTH or WI-FI, to the athlete's user electronic device (provided the user electronic device is within range) every 15 minutes. This data can then be sent, via cellular or wireless signal, to the computing system 150 for processing by the human performance tracking module 850.

### Human Performance Analysis

The biometric data from Table 6 can, for example, be used to establish unique athlete baseline averages over time for the metrics identified in Table 7. These may be calculated on a rolling average to account for training adaptations, such as shown in Table 7.

TABLE 7

| Metric | Units | Description |
|---|---|---|
| HR_{act} | Bpm | Heart rate during activity |
| HRᵣₑₛₜ | Bpm | Heart rate during rest |
| HRₛₗₑₑₚ | Bpm | Heart rate during sleep |
| HRR | Bpm | Heart rate recovery (per activity - HR >150 bpm |
| | | for 15 min. - calculated as HR_act minus HR 2 |
| | | minutes post) |
| HRVₛₗₑₑₚ | Ms | Heart rate variability, calculated by log- |
| | | transformed square root of the mean sum of the |
| | | squared differences between R-R intervals |
| MVT_{act} | Arbitrary | Movement during activity (combination of HR |
| | | and accelerometer data) |
| HVM | Arbitrary | High velocity movement |
| TL | Arbitrary | Training load (combination of MVT_{act} and |
| | | training duration) |
| MVTₛₗₑₑₚ | Arbitrary | Movement during sleep |
| RR_{act} | Breaths | Respiration rate during activity |
| | per min | |
| RRᵣₑₛₜ | Breaths | Respiration rate during rest |
| | per min | |
| RRₛₗₑₑₚ | Breaths | Respiration rate during sleep |
| | per min | |

The accelerometer in the biometric sensor 122 may be used to detect when a user is active (act), at rest or sleeping. Once the machine learning algorithm 1032 (FIG. 10) has acquired enough baseline data to understand the athlete's baseline for training, the RTM 1028 may use machine learning algorithms 1032 (FIG. 10) to identify trends and deviations from the baseline values in order to identify signs of nonfunctional overreaching and overtraining syndrome, as will be described in more detail in relation to FIG. 19.

The metric Movement Velocity Training (MVT) may have two different metrics. One MVT is determined during activity and is represented as MVT_{act}. The MVT_{act} may be determined using 3-D accelerometer and gyroscope data from the IMU to collect and analyze data on acceleration, frequency, duration, and intensity of movement. HR will not be used to determine MVT. Instead, HR should be used to calculate the TL (training load).

From a simplistic point of view, MVT may represent changes in posture and/or the related changing velocity.

The HVM metric may be based on amplitude of acceleration, frequency, duration and intensity data, and movement metrics that may be bucketed into a standard movement vector to denote walking by MVT and a high-velocity movement vector to denote running

The second MVT metric is determined during sleep activity and represented as MVTₛₗₑₑₚ. The MVTₛₗₑₑₚ may be determined by utilizing actigraphic methods (i.e., algorithms) by ActiGraph, LLC that analyze movement during sleep using the same data as activity tracking (acceleration, frequency, duration and intensity of 3D accelerometer and gyroscope data).

Specifically, the RTM 1028 may, for example, use machine learning algorithms 1032 to identify deviation patterns in the six (6) biometrics and/or calculated biometrics listed previously, as shown in Table 8.

TABLE 8

| | Deviation | |
|---|---|---|
| | Patterns | Description |
| 1 | Decreased | A decrease in HRV compared to baseline could |
| | HRVₛₗₑₑₚ | indicate overreach (OR) |
| 2 | Increased HRₛₗₑₑₚ | An increase in HR during sleep could indicate OR |
| 3 | Increased MVT_{act} | Increased sleep disturbances could indicate OR |
| 4 | Increased HRR | Increase in HRR that is associated with higher |
| | associated with | exertion (RR_{act}) indicates OR. Must discriminate |
| | increased RR_{act} | from Increased HRR associated with equal/lower |
| | | exertion, as that is a sign of positive adaption |
| 5 | Decreased HR_{act} | Decrease in HR_{act} that is associated with higher |
| | associated with | exertion (R_{act}) indicates OR. Must discriminate |
| | increased RR_{act} | from decreased HR_{act} associated with equal/lower |
| | | exertion, as that is a sign of positive adaption |
| 6 | Increased RR_{act} | An increase in exertion (RR_{act}) associated with an |
| | associated with | equal/lower training load could indicate OR |
| | equal or | |
| | decreased TL | |

Each of these deviation patterns may be normalized as a percentage of a baseline to determine a numeric score. The numeric score may be a health score. For example, the score for HRV is calculated based on equation EQ1 in the following way:

$$( \frac{H R {V}_{s l e e p} - H R {V}_{baseline}}{H R {V}_{baseline}} ) \times 100 = S c o r {e}_{H R V} \qquad EQ1$$

As an example, for the following values: HRVₛₗₑₑₚ=42 millesecond (ms) and HRV_{baseline}=75 ms, then the Score_{HRV} equals −44.

Similar calculations can be done for deviation patterns for the remaining monitored biometrics to determine an overall Overreaching Score defined by equation EQ2:

$$\frac{\begin{matrix}Scor {e}_{H R V} + S c o r {e}_{HR\_sleep} + S c o r {e}_{M V T} + \\ {Score}_{H R R} + S c o r {e}_{HR\_act} + S c o r {e}_{R R}\end{matrix}}{6} = S c o r {e}_{O v e r r e a c h i n g} \qquad EQ2$$

The Score_{HR_sleep} is determined by using the values of FIG. 7 into equation EQ1. Likewise, Score_{MVT}, Score_{HRR}, Score_{HR_act}, and Score_{RR} are determined using the values of FIG. 7 into equation EQ1. The equation EQ2 uses six Scores.

The equations may need to be altered slightly to account for directionality. For example, a decrease in HRV indicates NFOR, whereas and increase in HR indicates NFOR. As a result, the equation for HR must be (HR_baseline−HR_sleep)/(HR_baseline)×100 so that a negative adaptation is shown as a negative score.

Recall that the differentiation between functional overreaching and nonfunctional overreaching is the (1) intensity (2) consistency and (3) duration of symptoms. These can be measured in the following way, as shown in Table 9:

TABLE 9

| | Qualifier | Score_{Overreaching} Calculation |
|---|---|---|
| 1 | Intensity | Score_{Overreaching} absolute value exceeds threshold |
| 2 | Consistency | Range of 6 scores of EQ2 used to calculate |
| | | Score_{Overreaching} exceeds threshold |
| 3 | Duration | Number of consecutive days daily Score_{Overreaching} falls |
| | | outside standard deviation of rolling average exceeds |
| | | threshold |

If at least two of these qualifiers listed in Table 9 exceed their predetermined threshold for >1 week consecutively, an “NFOR Alert” can be triggered. If all three of these qualifiers in Table 9 exceed their predetermined threshold for >2 weeks, an “OTS Alert” may then be triggered. These alerts 1034 (FIG. 10) may be associated with athlete specific recommendations for how to better recover.

Over time, the machine learning algorithm 1032 (FIG. 10) can, for example, refine specific thresholds through learning both from the specific athlete and from the de-identified, aggregated database of athlete information.

Nonfunctional overreaching and overtraining syndrome may occur when a subject increases their physical training, for example, without the necessary rest to allow the body to recover. As is known in athletic training, nonfunctional overreaching may cause a short-term reduction in performance by the athlete. However, an athlete can often recover after a period of rest. In some scenarios, the athlete does not improve their performance by nonfunctional overreaching. However, the human performance tracking module 850 may guide the subject to functional overreaching which does lead to improved performance with a period of rest. Hence, the human performance training module 850 may provide sleep or rest recommendations 1036 (FIG. 10) that mitigates the nonfunctional overreaching condition so that the subject maintain a functional overreaching condition.

The human performance tracking module 850 may detect an overtraining syndrome and provide a training recommendation to recover. The overtraining syndrome may cause a reduction in performance Recovery from the overtraining syndrome may require a sustained period of rest.

The method blocks below may be performed in the order shown or a different order. One or more of the blocks may be performed contemporaneously. One or more blocks may be omitted or added.

FIG. 16 is a flowchart of an embodiment of a method 1600 for human performance tracking, such as provided by the human performance tracking module 850. The method 1600 may include, at block 1602, continuously collecting heart rate (HR) data from at least one sensor (i.e., biometric sensor device 122). The method 1600 may include, at block 1604, continuously collecting the resting rate (RR) data from at least one sensor (i.e., biometric sensor device 122). The method 1600 may include, at block 1606, continuously collecting activity data from at least one sensor (i.e., biometric sensor device 122).

The method 1600 may include, at block 1608, intermittently transmitting data to the athlete's electronic user device and human performance tracking (HPT) module. The method 1600 may include, at block 1610, storing the HR, RR and activity data from the one or more sensors. The method 1600 may include, at block 1612, analyzing and progressively creating baseline values for key metrics identified above in Tables 6 and 7 and ratios. As for ratios, if 5 vital signs are used as a basic case study, the normalized scale of 100 can be interpreted as a percentage. If HR, RR, BP, SpO2, TEMP at rest are all in the ideal range, a score of 100 results, for example. For a person just starting their training regimen, their initial TOTAL scores might start at 60. After 1 week, 70. After 1 month 85. This shows a trend towards an ideal range with a score of 100.

By way of non-limiting example, the human performance tracking metrics may be based on an “ideal” model. For example, if the subject plays basketball, the “ideal” model would be a function of performance metrics for an “ideal” basketball player, such as for jumping, running, etc. On the other hand, if the subject is a runner, the “ideal” model would be a function of performance metrics for an “ideal” runner or sprinter. The model may provide adjusted ranges for biometrics based on a particular human performance outcome yet to achieve.

From block 1612, the method 1600 may split between blocks 1614 and 1616. With regard to block 1614, a determination may be made whether the subject is sleeping. If the determination is “NO,” block 1614 may loop back to block 1602. If the determination is “YES,” block 1614 may proceed to FIG. 18, at block 1802 and block 1708 of FIG. 17. With regard to block 1616, a determination may be made whether the subject is training. If the determination is “NO,” block 1616 may loop back to block 1602. If the determination is “YES,” block 1616 may proceed to block 1702 of FIG. 17 and block 1904 of FIG. 19.

Returning again to block 1612, the method may proceed to block 1618. The method 1600, at block 1618 may include analyzing data compared to rolling baseline values for RHR, HRV, RR and activity to identify deviation patterns, as identified in Table 8. Activity may include sleep, rest or activity in the form of motion. The method 1600 may include, at block 1620, entering the baseline values into the overreaching score calculations, as describe above in relation to equation EQ1.

Returning again to block 1618, the method 1600 may include, at block 1622, looking up sleep quality data and recommendations 1036 from baseline values and human performance database. The method 1600 may include, at block 1624, displaying a recommendation 1036 based on the rolling baseline values. An example on a “rolling” baseline may include calculation over the past week (last 7 days) where today's baseline calculation replaces the value from 8 days ago, etc.

FIG. 17 is a flowchart of an embodiment of a method 1700 for training with nonfunctional, functional overreaching, and overtraining syndrome analysis. The method 1700 may include, at block 1702, tracking training sensor data during training. The method 1700 may include, at block 1704, analyzing the training data compared to the baseline values. The method 1700 may include, at block 1706, incorporating training data into the overreaching score equation EQ2. Baseline scores may be determined over a period of time (past week, past month, past 90 days, etc.) whereas the training scores are accumulated from the session or from the day. Baseline scores may be static rather than rolling. By way of non-limiting example, a baseline score may be chosen when the subject starts a particular training period, say at time T0. Time T0 may correspond to the time in which an implant device is implanted. The baseline score may be selected from those values taken at time T0 or over a first day or other initial time period.

The method 1700 may include, at block 1708, calculating overreaching score from key sleep and training metrics and compared to baseline values. The method 1700 may include, at block 1710, a determination may be made whether the overreaching score meets a daily threshold. If the determination is “NO,” the method 1700 proceeds to FIG. 16, block 1602. On the hand, if the determination is “YES,” the method 1700 may proceed to block 1712. The method 1700 may include, at block 1712, calculating an intensity, consistency and duration of the scores of Table 9. At block 1714, a determination may be made whether two of the three metrics of block 1712 are met. If the determination is “NO,” the method 1700 proceeds to FIG. 16, block 1602. On the hand, if the determination is “YES,” the method 1700 may proceed to block 1716. The method 1700 may include, at block 1716, tracking/logging consecutive days that meet the thresholds.

At block 1718, a determination can be made whether the nonfunctional overreach time threshold is met. If the determination is “NO,” the method 1700 proceeds to FIG. 16, block 1602. On the hand, if the determination is “YES,” the method 1700 may proceed to block 1720.

The method 1700 may include, at block 1720, displaying the nonfunctional overreach indicator and recommendation 1036 specific to the nonfunctional overreaching condition. The method 1700 may include, at block 1722, determining whether an overtraining syndrome time threshold is met. If the determination is “NO,” the method 1700 proceeds to FIG. 16, block 1602. On the hand, if the determination is “YES,” the method 1700 may proceed to block 1724. The method 1700 may include, at block 1724, displaying an overtraining syndrome indicator and recommendation 1036 specific to the overtraining syndrome. The method 1700 may end at block 1726.

FIG. 18 is a flowchart of an embodiment of a method 1800 for sleep tracking. The method 1800 may include, at block 1802, tracking sleep sensor data. Tracking the sleep sensor data may include storing and logging with timestamps one or more of the standard of care biometrics captured continuously from the implanted sensor system 120. The method 1800 may include, at block 1804, analyzing the sleep sensor data. The method 1800 may include, at block 1806, lookup sleep quality recommendations 1036 based on the analyzed sleep sensor data. The method 1800 may include, at block 1808, selectively displaying the sleep quality recommendation. The method 1800 may end at block 1810.

FIG. 19 is a flowchart of an embodiment of a method 1900 for training and recovery tracking. The method 1900 may include, at block 1904, continuously collecting key metrics from sensors, such as those associated from the implanted sensor system 120. The term “biometrics” is sometimes referred to as “metrics” herein. One or more of the HP key metrics correspond to cardiac function biometrics and biometrics derived from the cardiac function biometrics.

The HP key biometrics 1016 may include a single biometric (medical grade) or calculated biometrics derived from two or more biometrics (medical-grade). From a general population perspective, vital signs may be considered Human Performance key biometrics, as an example Each of the key metrics is a score associated with a biological system of the subject. The HP key biometrics 1016 may include training adaptation (TA) 1907A, exertion level (EL) 1907B, anaerobic threshold 1907C, metabolic lactate threshold 1907D, SpO₂ may be an indicator for altitude acclimation 1907E, training zones 1907F, endurance level 1907G and body temperature 1907H. Optionally, the HP key biometrics may include heart rate reserve. For example, the training adaptation (TA) 1907A may be determined based on blood pressure and heart rate. The exertion level (EL) 1907B may be calculated based on heart rate and resting rate. The metabolic lactate threshold 1907D will be described in more detail below. The metric SpO₂ (i.e., altitude acclimatization) 1907E is determined. The training zones 1907F is determined by heart rate and SpO₂. The endurance level 1907G may be determined by HRR. The body temperature 1907H is measured by the sensor device 122 directly for example

A potential indicator of dehydration may include a high hematocrit level. A comprehensive metabolic panel may be used for nutrition, muscle status and inflammation. Bicarbonate loading may provide an indicator representative of performance enhancing effects. Creatinine combined with BUN may represent dehydration. Glucose may be used for sports nutrition planning and intake during or after a training event. Sodium, combined with chloride, glucose, bicarbonate and hematocrit may provide a measure representative of serum osmolality or dehydration. Temperature may drive heat acclimatization efforts to improve performance Measurements of the blood pressure may be used as a metric to define a rate of recovery. Blood pressure may represent symptomatic hypotension and syncope.

A table representative of training intensity zone metrics is shown in Table 10.

TABLE 10

| Intensity | VO₂ | Heart Rate | Lactate | Duration | |
|---|---|---|---|---|---|
| Zone | (% max) | (% max) | (mmd · L⁻¹) | Within Zone | |
| 1 | 45-65 | 55-75 | 0.8-1.5 | 1-6 | Hours |
| 2 | 66-80 | 75-85 | 1.5-2.5 | 1-3 | Hours |
| 3 | 81-87 | 85-90 | 2.5-4 | 50-90 | min. |
| 4 | 88-93 | 90-95 | 4-6 | 30-60 | min. |
| 5 | 94-100 | 95-100 | 6-10 | 15-30 | min. |

A table representative is shown in Table 11.

| HP Key | Biometric | |
|---|---|---|
| Biometrics | Parameters Used | Description |
| Training | BP, HR, | During activity (as determined by accelerometer |
| Adaptation | accelerometer, | and HR data), key biometrics will be tracked |
| (1907A) | SpO2, RR | continuously and time stamped. Exertion level |
| | | (1907B) will be time stamped along with, HR BP |
| | | and SpO₂. Rolling 30-day averages for key ratios |
| | | (HR/exertion, BP/exertion, and SpO₂/exertion) will |
| | | be calculated and stored in the athlete's profile. |
| | | Deviations from the averages for these key ratios |
| | | will indicate training adaptation. For example, |
| | | lower HR/exertion, lower BP/exertion and higher |
| | | SpO2/exertion all indicate positive adaptation. |
| | | Trends in these metrics will be identified and |
| | | displayed for the user along with training |
| | | recommendations based on trends. |
| Exertion Level | HR, | Respiratory rate (RR) measured in breaths per |
| (1907B) | accelerometer | minute, will be collected during an activity (as |
| | and respiratory | determined by accelerometer and HR data). At the |
| | rate (RR) | end of the workout, RR_max and RR_average for |
| | | the activity will be calculated and displayed. |
| | | RR_max is a peak value. RR_average is an |
| | | average of RR over a determined period of time. |
| | | Additionally, the RR/HR ratio will be calculated |
| | | and time stamped throughout the activity. This |
| | | information will be displayed for the activity and |
| | | incorporated into the baseline RR/HR ratio for that |
| | | athlete. Trends and deviations in this ratio will be |
| | | used to further identify increasing exertion levels |
| | | for given physiological output (higher-than-normal |
| | | RR/HR indicates abnormally high exertion and |
| | | potential fatigue. Lower-than-normal RR/HR |
| | | indicates positive training adaptation and |
| | | physiological output capacity surplus. |
| Anaerobic | HR, RR, | During activity, time stamped training load |
| Threshold | accelerometer, | intensity will be determined by a combination of |
| (1907C) | SpO₂ | HR, RR and accelerometer data. The corresponding |
| | | SpO₂ levels at these various intensities will be |
| | | collected and recorded. An individualized profile |
| | | for SpO₂ levels and respective activity loads will be |
| | | analyzed and developed. |
| | | Anaerobic threshold is the training load at which |
| | | there in a downward inflection point in % SpO₂ |
| | | levels (and upward inflection point for lactate |
| | | levels - see 1907D below). Through continuously |
| | | monitoring % SpO₂ levels, the system can identify |
| | | the specific training load at which this inflection |
| | | point occurs and record the correlated load |
| | | intensity, HR and RR. This data will be displayed |
| | | for the athlete and logged to be calculated into the |
| | | athlete's profile and baseline anaerobic threshold. |
| | | The system will identify trends in anaerobic |
| | | threshold. If, in subsequent activities, the anaerobic |
| | | inflection point occurs at a higher training load |
| | | intensity, this is indicative of positive adaptation to |
| | | training. Conversely, lower anaerobic thresholds |
| | | may indicate negative training adaptations and/or |
| | | fatigue |
| Metabolic | HR, RR, | See above. The process may leverage lactate |
| Lactate | accelerometer, | concentration at various training intensity loads to |
| Threshold | metabolic | determine positive inflection point, indicating |
| (1907D) | lactate | anaerobic threshold. |
| | concentration | Can be used in conjunction with SpO₂-based |
| | | calculation to provide further accuracy and insight |
| | | into calculation. |
| Altitude | Accelerometer, | Measure SpO₂ levels at rest, ideally while sleeping |
| Acclimatization | SpO₂ | (based on accelerometer data). Establish baseline |
| (1907E) | | SpO₂ level for individual athletes. Increase in |
| | | altitude will lead to decrease in resting SpO₂ levels, |
| | | which will lead to decreased athletic performance. |
| | | Gradual acclimatization will result in gradual |
| | | increase of resting SpO₂ levels back to baseline. |
| | | System will identify dramatic drops in SpO₂ and |
| | | identify potential increases in altitude. System will |
| | | then display daily SpO₂ values for athlete to |
| | | illustrate gradual increase associated with |
| | | acclimatization. Can be used to identify current |
| | | level of acclimatization, time estimates until fully |
| | | acclimated, and training recommendations based |
| | | on stage of acclimatization. |
| Training Zones | Accelerometer, | There are 5 commonly accepted training zones (see |
| (1907F) | HR, RR, SpO₂, | chart below). Training within a desired “training |
| | lactate | zone” is imperative for achieving specific goals of |
| | | training. Percent of HR_max is widely used; |
| | | however it is less precise. More precise |
| | | measurement factors in O2, lactate and activity |
| | | duration as well. |
| | | This system will continuously track/collect data for |
| | | these key metrics during activity. This data will be |
| | | factored into an athlete's profile and baseline for |
| | | various zones. For each subsequent activity, the |
| | | data collected will be compared to the athlete's |
| | | baseline to determine specifically which zone they |
| | | were training within. |
| | | The system will also look for deviations from the |
| | | aggregated baseline to identify positive or negative |
| | | adaptations to training. For example, if lower |
| | | values for these metrics are recognized for |
| | | activities of similar load intensity and duration, this |
| | | can be indicative of positive training adaptation and |
| | | a shift in that athletes individualized training zones, |
| | | in Table 10. |
| Endurance | Accelerometer, | Heart rate recovery is calculated by measuring the |
| Level using | RR (respiration | difference between HR during exercise and HR 1, 2 |
| HRR (heart | rate), HR | and 3 minutes immediately post exercise (HRR1, |
| rate recovery) | | HRR2, and HRR3). Units are BPM. |
| (1907G) | | High HRR (i.e., rapid recovery to normal HR) is |
| | | indicative of high cardiovascular conditioning. |
| | | Recognizing trends will be key for this utility. |
| | | However, deviations of lower HRR from average |
| | | can indicate high fatigue/low recovery from |
| | | previous workout and should be factored into |
| | | NFOR/OTS calculations. |
| Temperature | ° F. | Body Temperature |
| (1907H) | | |
| Heart Rate | Accelerometer, | Classic Karvonen Formula for heart rate recovery |
| Reserve | HR | is typically an estimate based on your HR (max) − |
| (optional) | | HR (rest). HR (max) can be estimated by |
| | | subtracting your age from 220 and determining |
| | | your HR @ rest. Say for a typical, reasonably fit |
| | | 50 year old with HR @ rest = 50 bmp, HRR = (220 − |
| | | 50) − 50 = 120. Karvonen Formula has been |
| | | refined to 206.9 − (0.67 × age) = 206.9 − (0.67 × |
| | | 50) = 173.4 for our 50 year old, reasonably fit |
| | | example. |
| | | For embodiments herein, the HRR is determined |
| | | based on the individual's performance and |
| | | determine on a time based number (i.e., Past week, |
| | | past month, past 90 days, past year, etc.) by |
| | | determining the actual HR max over the given time |
| | | period and the HR rest over the same given time |
| | | period. |
| | | Example: if over the past 30 days HR max (this |
| | | can be a peak number, or a rolling average say of |
| | | the top 5 peak values over the past 30 days) is 200 |
| | | bpm and the HR rest (this can be a minimum |
| | | number, or a rolling average say of the 5 minimum |
| | | values) is 50 bpm, the HRR is 200 − 50 = 150. |

By way of non-limiting example, the respiratory rate alone may indicate an exertion level. The respiratory rate when paired with the heart rate, an indication of increased exertion level associated with higher physiological output may be determined, for example. Alternately, an increased exertion level may be associated with lower physiological output representative of fatigue, for example.

The metabolic lactate inflection point (i.e., lactate threshold) may be represented by the exercise intensity at which the blood concentration of lactate and/or lactic acid begins to increase exponentially. It is often expressed as 85% of maximum heart rate or 75% of maximum oxygen intake, se measured in real-time by the biometric sensor device 122.

By way of non-limiting example, altitude acclimatization may be based on relative SpO₂ levels that may be monitored for large inflections followed by gradual recovery to indicate a change in altitude change and acclimatization. SpO₂ levels (as compared to rolling one month average) should be used to indicate acclimatization. An initial drop in SpO₂ may, for example, be expected, followed by a progressing recovery over 2-4 weeks to athlete's 30-day rolling average SpO2 level indicates where in the acclimatization process an athlete may be.

The method 1900 may include, at block 1908, determining whether the body temperature is in a standard range. If the determination is “NO,” the method 1900 may trigger an alert to the user electronic device of the out-of-range temperature condition, at block 1910. If the determination is “YES,” the method 1900 may begin time stamping collected biometric data throughout training, at block 1912.

The determination made at block 1908 may be repeated for each specific key biometric 1016. In other words, if any particular metric is out-of-range by a certain threshold from the baseline values, an alert may be generated and displayed by the user electronic device. In some embodiments, an alert may include an audible alert indicator.

The method 1900 may include, at block 1914, transmit biometric data, collected by the implanted sensor system 120, at end of training session to the computing system 150. The method 1900 may include, at block 1916, store biometric sensor data, and analyze the data to develop baseline values for HP key metrics. The method 1900 may include, at block 1918, selectively display statistics from the workout on the user electronic device via application 157. Examples of displayed statistics is shown in FIG. 20B. The baseline values may be generated after the first use or over a course of several initial training routines. After the baseline values are collected, the application 157 may switch from baseline collection to monitoring/tracking thereafter.

The method 1900 may include, at block 1920, comparing the baseline values and trend analysis based on stored biometric data (i.e., key metrics 1016) captured during training. The method 1900 may include, at block 1922, look up training recommendations based on workout statistics and trend identification (ID). The method 1900 may include, at block 1924, displaying trending recommendations on the user electronic device via the application 157, as will be described in relation to FIGS. 20A and 20B.

FIG. 20A is a diagram of an embodiment of a mobile device displaying a human performance tracking GUI 2000A. The human performance tracking GUI 2000A may display one or more of the biometrics described above in relation to Tables 6-9 and calculations associated with equations EQ1 and EQ2. For the sake of illustration, the GUI 2000A displays the following biometrics HRV, RHR, functional overreaching, nonfunctional overreaching, overtraining syndrome, sleep, altitude acclimation, RR, endurance level, exertion level, training adaptation, anabolic threshold, training zones, and metabolic lactate, for example. Each biometric may be individually selectable via radio buttons or other selection tools, using a touch screen user interface of the display.

The human performance tracking GUI 2000A may include navigational tools for selecting and displaying activity biometrics using the activity button 2010. The biometric data is collected for each training session and/or during sleep. The user may be able to display any of the biometrics capture during sleep separately from those metrics captured during training, as will be described in relation to FIG. 20B.

FIG. 20B is a diagram of an embodiment of a mobile device displaying another human performance tracking GUI. For the sake of illustration, assume that the biometrics HRV and RHR has been selected using GUI 2000A of FIG. 20A, as denoted by the black shading of the radio buttons.

The human performance tracking GUI 2000B may include a similar navigation tools as described above in relation to FIG. 11. The human performance tracking GUI 2000B may include buttons 2016 for recovery, button 2018 for tracking training and button 2019 for tracking sleeping. For example, before a user begins a training routine, the user may select the button 2018 so that the biometrics collected thereafter are recorded for activities or training. On the other hand, selecting the button 2019 before a user begins sleeping, allows the biometrics collected to be recorded for sleeping. Still further, selecting the recovery button 2016, allows the biometrics collected thereafter to be recorded as recovery, until another button (i.e., buttons 2018 or 2019) is selected, in some examples. In other examples, the activity sensor 804 (i.e., IMU) may detect a level of motion to indicate training or sleeping. The IMU may identify low intensity motion or non-training motion such as, without limitation, walking, climbing stairs, etc.

The human performance tracking GUI 2000B may include tracked performance metrics. For example, the human performance tracking GUI 2000 may display a chart 2020 or graphs of HRV over a period of time. In the illustration, the period of time is a one-week interval. However, other increments of time may be displayed based on a user selection. The GUI 2000B may include a current HRV reading 2022 and/or a percentage of change 2024.

For example, the human performance tracking GUI 2000B may display a chart 2030 of RHR over a period of time. In the illustration, the period of time is a one-week interval. However, other increments of time may be displayed based on a user selection. The human performance tracking GUI 2000B may include a current RHR reading 2032 and/or a percentage of change 2034.

The human performance tracking GUI 2000B may include an e-coaching field 2040 configured to display information associated with a recommendation to improve the physical condition of the subject or to improve training and recovery.

The performance tracking is not limited to athlete training. The performance tracking may be used to access the biological system of a subject or patient undergoing a rehabilitation routine, such as during activity associated with physical therapy, occupational therapy and the like, intended to improve a subject's ability to perform activity.

The performing tracking may include tracking basic metabolic panel biometrics. For example, deficiencies in sodium or potassium may be depleted based on certain training routines and the level of exertion. According, as these biometrics become depleted, the application 157 may provide alerts to recommend the need to take certain supplements or vitamins to raise levels of depleted biometrics. The example of sodium and potassium are meant to be illustrative and not intended to be limiting. Any of the continuously sensed biometrics which are out-of-range may cause the application 157 to generate an alert and/or recommendation.

It should be understood that any of the scores for health or performance are biological system scores that may use continuously sensed biometrics are part of the inputs for calculating a score.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques or one or more blocks of the methods may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media that corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques and blocks of methods herein. Also, the techniques could be fully implemented in one or more circuits or logic elements.

FIG. 21 depicts an example of internal hardware that may be included in any of the electronic components of an electronic device as described in this disclosure such as, for example, an on-premises electronic device, an associate electronic device, a remote electronic device and/or any other integrated system and/or hardware that may be used to contain or implement program instructions.

A bus 2100 serves as the main information highway interconnecting the other illustrated components of the hardware. CPU 2105 is the central processing unit of the system, performing calculations and logic operations as may be required to execute a program. CPU 2105, alone or in conjunction with one or more of the other elements disclosed in FIG. 21, is an example of a processor as such term is used within this disclosure. Read only memory (ROM) and random access memory (RAM) constitute examples of tangible and non-transitory computer-readable storage media 2120, memory devices or data stores as such terms are used within this disclosure. The memory device may store an operating system (OS) of the server or for the platform of the electronic device.

Program instructions, software or interactive modules for providing the interface and performing any querying or analysis associated with one or more data sets may be stored in the computer-readable storage media 2120. Optionally, the program instructions may be stored on a tangible, non-transitory computer-readable medium such as a compact disk, a digital disk, flash memory, a memory card, a universal serial bus (USB) drive, an optical disc storage medium and/or other recording medium.

An optional display interface 2130 may permit information from the bus 2100 to be displayed on the display 2135 in audio, visual, graphic or alphanumeric format. Communication with external devices may occur using various communication ports 2140. A communication port 2140 may be attached to a communications network, such as the Internet or an intranet 115 (FIG. 1B). In various embodiments, communication with external devices may occur via one or more short range communication protocols. The communication port 2140 may include communication devices for wired or wireless communications.

The hardware may also include an interface 2145, such as graphical user interface (GUI), that allows for receipt of data from input devices such as a keyboard or other input device 2150 such as a mouse, a joystick, a touch screen, a remote control, a pointing device, a video input device and/or an audio input device. The GUIs, described herein, may be displayed using a browser application being executed by an electronic device and served by a server of the system 100B. For example, hypertext markup language (HTML) may be used for designing the GUI with HTML tags to the images of the assets and other information stored in or served from memory 155.

In this document, “electronic communication” refers to the transmission of data via one or more signals between two or more electronic devices, whether through a wired or wireless network, and whether directly or indirectly via one or more intermediary devices. Devices are “communicatively connected” if the devices are able to send and/or receive data via electronic communication.

The features and functions described above, as well as alternatives, may be combined into many other different systems or applications. Various alternatives, modifications, variations or improvements may be made by those skilled in the art, each of which is also intended to be encompassed by the disclosed embodiments.

What is claimed is:

1. A system, comprising:
at least one implanted sensor device comprising one or more continuous sensors configured to detect a first modular set of biometrics of a subject;
an assessment system comprising:
a computing device, and
a computer-readable storage medium comprising one or more programming instructions that, when executed, cause the computing device to:
receive the first modular set of biometrics from the one or more continuous sensors over a period of time,
receive a second modular set of biometrics associated with the subject,
selectively serve a graphical user interface configured to present one or more of the first modular set of biometrics and the second modular set of biometrics,
normalize at least a portion of the first modular set of biometrics and the second modular set of biometrics over a period of time,
generate a score indicative of a state of a biological system of the subject corresponding to the at least a portion of biometrics of the first modular set of biometrics and the second modular set of biometrics, and
cause the score to be displayed via a client electronic device.

2. The system of claim 1, wherein the one or more continuous sensors comprise one or more of the following:
an inertial measurement unit;
an electrocardiogram sensor;
a photoplethysmogram;
a thermometer; or
a microphone.

3. The system of claim 1, wherein the second modular set of biometrics comprises one or more of the following:
a lipid panel biometrics;
a basic metabolic panel biometrics;
a comprehensive metabolic panel biometrics; or
infection biometrics.

4. The system of claim 1, wherein the at least one implanted sensor device continuously detects a heart rate, a blood pressure, a temperature, oxygen saturation, respiration and activity of the subject.

5. The system of claim 4, wherein the activity is tracked by activity categories comprise one or more of the following:
sleeping;
non-training motion;
resting; or
training.

6. The system of claim 5, wherein the computer-readable storage medium further comprises one or more programming instructions that, when executed, cause the computing device to determine human performance of the subject based on one or more of the following:
training adaptation;
exertion level;
anaerobic threshold;
metabolic lactate threshold;
altitude acclimation;
endurance level; or
temperature.

7. The system of claim 5, wherein the computer-readable storage medium further comprises one or more programming instructions that, when executed, cause the computing device to determine human performance of the subject based on one or more of the following:
heart rate recovery; or
heart rate reserve.

8. The system of claim 5, wherein the computer-readable storage medium further comprises one or more programming instructions that, when executed, cause the computing device to determine one or more of the following:
functional overreaching;
nonfunctional overreaching; or
early signs of an overtraining syndrome.

9. The system of claim 4, wherein the at least one implanted sensor device comprising one or more sensors to continuously detect a basic metabolic panel biometrics and cardiac function biometrics.

10. The system of claim 1, wherein the first modular set of biometrics and the second set of biometrics are medical-grade biometrics, wherein the computing device configured to interface with a non-medical grade continuous sensor device and receive non-medical grade sensor biometric data; and the score is determined in part based on the non-medical grade continuous sensor device.

11. A method, comprising:
sensing, by at least one implanted sensor device comprising one or more continuous sensors, a first modular set of biometrics of a subject; and
by at least one processor:
receiving the first modular set of biometrics from the one or more continuous sensors over a period of time,
receiving a second modular set of biometrics associated with the subject,
selectively serving a graphical user interface configured to present one or more of the first modular set of biometrics and the second modular set of biometrics,
normalizing at least a portion of the first modular set of biometrics and the second modular set of biometrics over a period of time,
generating a score indicative of a state of a biological system of the subject corresponding to the at least a portion of biometrics of the first modular set of biometrics and the second modular set of biometrics, and
causing the score to be displayed via a client electronic device.

12. The method of claim 11, wherein the one or more continuous sensors comprise one or more of the following:
an inertial measurement unit;
an electrocardiogram sensor;
a photoplethysmogram;
a thermometer; or
a microphone.

13. The method of claim 11, wherein the second modular set of biometrics comprises one or more of the following:
a lipid panel biometrics;
a basic metabolic panel biometrics;
a comprehensive metabolic panel biometrics; or
infection biometrics.

14. The method of claim 11, wherein the sensing comprises continuously detecting:
a heart rate;
a blood pressure;
a temperature;
an oxygen saturation;
a respiration; and
activity of the subject.

15. The method of claim 14, further comprising:
tracking the activity by activity categories comprise one or more of the following:
sleeping;
non-training motion;
resting; or
training.

16. The method of claim 15, wherein the generating of the score comprises generating a metric score for one or more of the following:
training adaptation;
exertion level;
anaerobic threshold;
metabolic lactate threshold;
altitude acclimation;
endurance level; or
temperature.

17. The method of claim 16, wherein the generating of the score comprises generating a score for one or more of the following:
functional overreaching;
nonfunctional overreaching; or
early signs of overtraining syndrome.

18. The method of claim 11, wherein the sensing comprises continuously sensing a basic metabolic panel biometrics and cardiac function biometrics.

19. The method of claim 11, wherein the first modular set of biometrics and the second set of biometrics are medical-grade biometrics.

20. The method of claim 19, further comprising:
interfacing with a non-medical grade continuous sensor device; and
receiving non-medical grade sensor biometric data wherein the score is determined in part based on the non-medical grade continuous sensor device.

21. The method of claim 19, further comprising:
receiving alert level instructions for a medical professional associated with the score; and
determining that the score meets the alert level instructions; and
automatically communicating to the medical profession an alert associated with the alert level instructions, in response to determining that the score meets the alert level instructions.
